(19) United States
(12) Patent Application Publication
(10) Pub. No.: US 2024/0307394 A1
(43) Pub. Date: Sep. 19, 2024

(54) MODULATORS OF MAS-RELATED G-PROTEIN RECEPTOR D AND RELATED PRODUCTS AND METHODS

(71) Applicant: ESCIENT PHARMACEUTICALS, INC., San Diego, CA (US)

(72) Inventors: Marion Lanier, San Diego, CA (US); Marcus Boehm, San Diego, CA (US); Esther Martinborough, San Diego, CA (US)

(21) Appl. No.: 18/607,196

(22) Filed: Mar. 15, 2024

Related U.S. Application Data
(60) Provisional application No. 63/491,003, filed on Mar. 17, 2023.

(51) Int. Cl. A61K 31/506; C07D 239/47; C07D 239/52; C07D 401/12

(52) U.S. Cl. CPC A61K 31/506; C07D 239/47; C07D 239/52; C07D 401/12

(57) ABSTRACT

Compounds, pharmaceutical compositions thereof, and methods are provided for modulating MRGPR D generally, or for treating a MRGPR D dependent condition more specifically, by contacting the MRGPR D or administering to a subject in need thereof, respectively, an effective amount of a compound having the structure of Formula (1):

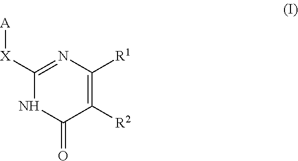

or a pharmaceutically acceptable isomer, racemate, hydrate, solvate, isotope, or salt thereof, wherein A, X, R¹ and R² is as defined herein.

### BACKGROUND

### Technical Field

The invention relates to modulators of the Mas-related G-protein coupled receptor D (MRGPRD), to products containing the same, as well as to methods of their use and preparation.

### Description of the Related Art

Mas-related G protein receptors (MRGPRs) are a group of orphan receptors with limited expression in very specialized tissues. Little is known about the function of most of these receptors. There are eight related receptors in this class expressed in humans, only four of which have readily identifiable orthologs in other species (i.e., MRGPR D, E, F and G). Some of the other four receptors (MRGPR X1, X2, X3 and X4) have counterparts in higher species including dogs and horses, but they do not have a single corresponding ortholog in rodents.

### BRIEF SUMMARY

This invention is based, in part, on the identification of MRGPRD or MRGPRD ortholog modulator compounds. MRGPRD corresponds functionally to mouse and rat Mrgprd. MRGPRD and its orthologs are expressed in the dorsal root ganglia as well as several peripheral organs. MRGPRD and its orthologs have been shown to be involved in pain signaling, physiological and pathophysiological processes of the gastrointestinal (GI) tract, Ca²⁺ dysregulation in the heart (cardiac output and vascular tone) and have also been shown to be expressed in skin, immune cells, the eye, kidney, and brain.

Accordingly, in some embodiments, compounds are provided having the structure of Formula (I):

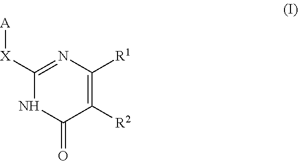

or a pharmaceutically acceptable isomer, racemate, hydrate, solvate, isotope, or salt thereof, wherein A, X, R¹ and R² are as defined below.

In one embodiment, compounds are provided having the structure of Formula (II):

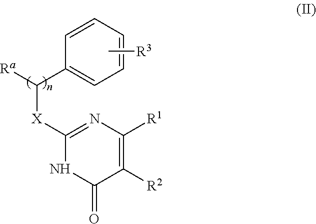

or a pharmaceutically acceptable isomer, racemate, hydrate, solvate, isotope, or salt thereof, wherein X, R^{a}, R¹, R² and R³ are as defined below.

In one embodiment, compounds are provided having the structure of Formula (IIa):

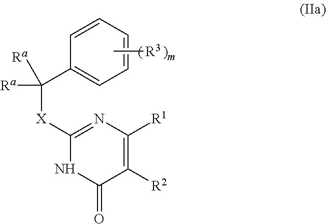

or a pharmaceutically acceptable isomer, racemate, hydrate, solvate, isotope, or salt thereof, wherein X, R^{a}, R¹, R², R³ and m are as defined below.

In one embodiment, compounds are provided having the structure of Formula (III):

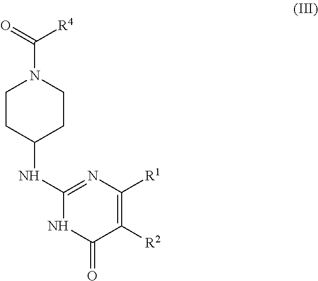

or a pharmaceutically acceptable isomer, racemate, hydrate, solvate, isotope, or salt thereof, wherein R¹, R² and R⁴ are as defined below.

In one embodiment, compounds are provided having the structure of Formula (IIIa):

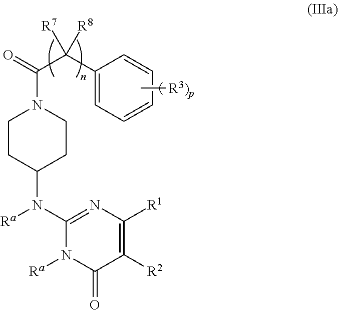

or a pharmaceutically acceptable isomer, racemate, hydrate, solvate, isotope, or salt thereof, wherein R^{a}, R¹, R², R³, R⁷, R⁸, n and p are as defined below.

In one embodiment, compounds are provided having the structure of Formula (IV):

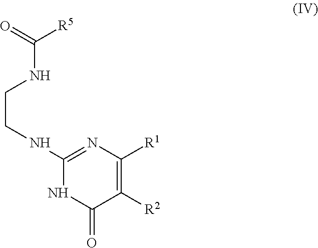

or a pharmaceutically acceptable isomer, racemate, hydrate, solvate, isotope, or salt thereof, wherein R¹, R² and R⁴ are as defined below.

In one embodiment, compounds are provided having the structure of Formula (IVa):

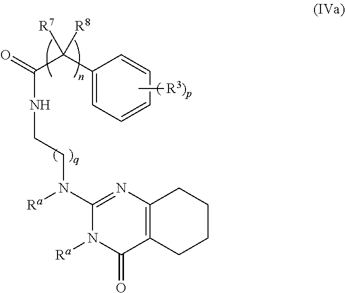

or a pharmaceutically acceptable isomer, racemate, hydrate, solvate, isotope, or salt thereof, wherein R^{a}, R³, R⁷, R⁸, n, p and q are as defined below.

In another embodiment, pharmaceutical compositions are provided comprising a compound having the structure of Formula (I), (II), (IIa), (III), (IIIa), (IV) or (IVa), or a pharmaceutically acceptable isomer, racemate, hydrate, solvate, isotope, or salt thereof, in combination with a pharmaceutically acceptable excipient.

In another embodiment, methods are provided for modulating a MRGPR D by contacting the MRGPR D with an effective amount of a compound having the structure of Formula (I), (II), (IIa), (III), (IIIa), (IV) or (IVa), or a pharmaceutically acceptable isomer, racemate, hydrate, solvate, isotope, or salt thereof.

In another embodiment, methods are provided for treating a MRGPR D dependent condition by administering to a subject in need thereof an effective amount of a compound having the structure of Formula (I), (II), (IIa), (III), (IIIa), (IV) or (IVa), or a pharmaceutically acceptable isomer, racemate, hydrate, solvate, isotope, or salt thereof.

In certain embodiments, the MRGPR D dependent condition is a pain associated condition, an itch associated condition, an ocular associated condition, a cardiovascular and renal disease associate condition, an inflammatory or autoimmune disorder, or a cognitive impairment associated condition, a cancer related condition, or a non-cancerous hyperproliferative disorder.

In more specific embodiments, the MRGPR D dependent condition is one or more of a dry eye syndrome/keratoconjunctivitis sicca and related conditions, chronic itch (e.g., pruritus), inflammation disorders, autoimmunity, skin disorders, cardiovascular disease, renal disease, treat cognitive impairment due to neurodegenerative diseases, age-induced cognitive impairment, vascular cognitive impairment, post-stroke cognitive impairment, and psychiatric disorders.

In further embodiments, prodrugs and/or metabolites of a compound having the structure of

Formula (I), (II), (IIa), (III), (IIIa), (IV) or (IVa), are also provided. In the case of prodrugs, a compound (i.e., prodrug) may be administered to a subject which is then converted in vivo to a compound having the structure of Formula (I), (II), (IIa), (III), (IIIa), (IV) or (IVa). In the case of metabolites, following administration to a subject of a compound having the structure of Formula (I), (II), (IIa), (III), (IIIa), (IV) or (IVa) such compound may be converted in vivo to an active metabolite.

### DETAILED DESCRIPTION

Mas-related G-protein-coupled receptors (MRGPRs) comprise a subfamily of Class A receptors named after the first discovered member, Mas. MRGPRs were first identified on specialized sensory neurons that encode itch (pruriceptors) and pain (nociceptors). Both neuronal subtypes have their cell bodies residing in the dorsal root and trigeminal ganglia (DRG and TG). Importantly, activation of Mrgprs expressed at the surface of sensory neurons has been shown to induce both non-histaminergic itch, pain sensations and mechanical/visceral hypersensitivity.

The eight human MRGPRs comprise of MRGPRX1-4 (1,2,3 and 4) and MRGPRD-G (D, E, F, and G). Whilst MRGPRX1-4 are only expressed in humans and some higher species, MRGPRD-G seem to be expressed in all mammals. The orthologs of MRGPRD are readily identified despite low sequence homology. Of note, MRGPRD is encoded by a single copy MRGPRD gene with defined orthologues in rodents and humans and thus constitutes an attractive therapeutic target for pain and other indications.

Despite the sensory nature of these receptors, the function of these MRGPRs has proved elusive. Trianglulation of agonist profiles, receptor expression in tissues and loss/gain of function in animal (disease) models has proven important in recognizing the therapeutic potential of these receptors.

The first ligand described for MRGPRD of human, rat, mouse, and monkey was B-alanine. This amino acid analog is produced in the liver from uracil or from dietary carnosine by the enzyme carnosinase. Coupling to calcium-activated chloride channels via Gq proteins, phospholipase C, and inositol-3 phosphate-induced Ca²⁺ release has been described.

Alamandine, Ala1-Ang-(1-7), a peptide in the Renin-Angiotensin System (RAS), has also been described as an agonist of MRGPRD. Peptides seem to interact differently with the receptor than β-alanine, because B-alanine does not inhibit the vasodilatory actions of alamandine. However, D-Pro7-Ang-(1-7) was shown to be an inhibitor of alamandine binding to MRGPRD.

5-oxoETE, a lipid elevated in the gut of IBS patients, has been shown to induce calcium signaling in sensory neurons that is dependent on the presence of MRGPRD, suggesting that 5-oxoETE might signal in neurons via MRGPRD.

In addition to DRG, the expression of MRGPRD transcripts has been identified in several peripheral organs including artery, heart, bladder, GI tract, eye, brain, and kidney. This suggests that MRGPRD may have a role in several peripheral indications.

MRGPRD is expressed at very high levels in most unmyelinated nociceptive neurons that are labeled by isolectin-B4 and expressed in DRG. Like other members in the MRGPRs family, MRGPRD has been suggested to be highly related to the sensation of pain and itch.

MRGPRD activation has been shown to mediate pain signaling, characterized by hypersensitivity to multiple stimuli that lead to painful sensation once integrated in the brain. Indeed, elevated expression of MRGPRD is observed in models of neuropathic pain.

Expression of MRGPRD has been demonstrated in sensory neurons innervating the colon. In colon-projecting sensory neurons, 41% of TRPV1-positive neurons were also reported to express MRGPRD. Activation of MRGPRD signaling in the colon has been shown to participate in the development of pain sensation in the context of irritable bowel syndrome (IBS).

The expression of MRGPRD in the enteric neurons suggests that MRGPRD is highly associated with physiological and pathophysiological processes of the GI tract, such as bowel motility/dysmotility and intestinal inflammation.

In a recent study, an increase in the arachidonic acid metabolite 5-oxoETE was found in biopsies from patients with clinically established IBS compared to healthy subjects. 5-oxoETE has been shown to induce calcium signaling in sensory neurons. In the absence of MRGPRD, activation of sensory neurons by 5-oxoETE was significantly decreased, suggesting that 5-oxoETE might signal in neurons via MRGPRD.

In the aorta/heart, MRGPRD has been reported to be activated by alamandine, and to produce endothelial-dependent vasodilation in rat and mouse aortic rings. In hypertensive rats, alamandine treatment restored the contractile function and prevented Ca²⁺ dysregulation via activation of MrgprD in cardiomyocytes.

Alamandine has also been reported to act via MRGPRDto induce AMPK/NO signaling to counter-regulate ANGII-induced hypertrophy, highlighting the therapeutic potential of the alamandine/MrgD axis in the heart.

Blockers of the receptors of alternate RAS, such as the MRGPRD, increase splanchnic vascular resistance in cirrhotic animals, and thus drugs targeting the alternate RAS may be useful in the treatment of portal hypertension and liver fibrosis.

MRGPRD has been found to be expressed in neutrophils and is thought to be involved in inflammatory reactions.

Alamandine, through MRGPRD receptors, do not affect MO macrophages but reduce the proinflammatory TNF-α, CCL2, and IL-1β transcript expression levels in LPS+IFN-γ-stimulated macrophages.

MRGPRD is expressed in retinal neurons, retinal vasculature, Müller glial and RPE cells. MRGPRD-deficient mice do not exhibit gross changes in retinal morphology and thickness in aging. In vitro studies in human retinal cells show that alamandine attenuated increases in inflammatory cytokine gene expression and production of reactive oxygen species. These results support the notion that alamandine/MRGPRD may represent another new protective axis of RAS in the retina exerting anti-oxidative and anti-inflammatory effects.

Allantoin induces scratching behavior in mouse model of chronic kidney disease and activates DRG neurons; the calcium influx and the action potential were significantly reduced in DRG neurons of MRGPRD KO mice, suggesting a role for MRGPRD in chronic kidney disease (CKD).

Studies in the mouse brain show that MRGPRD-positive cells have been identified in some forebrain areas, including cortex, hippocampus, amygdala, hypothalamus, habenular nuclei, striatum and pallidum, as well as in some mid-brain nuclei in a region-specific manner. The specific localization of MRGPRD in the reward-and limbic-related areas can hint at a role of MRGPRD in processes such as pain perception/modulation, synaptic plasticity, learning, memory, and cognition.

Additionally, alamandine induces antidepressant-like effects in low brain angiotensinogen transgenic rats. An MRGPRD receptor antagonist reversed the antidepressant-like effect induced by alamandine, suggesting a role of MRGPRD in the treatment of neuropsychiatric diseases.

MRGPRD is reported to be expressed in lung cancer and to promote tumorigenesis. Therefore, targeting MRGPRD may provide a novel therapy for lung or other cancers.

In some embodiments, a method for the treatment of an MRGPRD dependent cancer related condition is provided, the method comprising administering an effective amount of a compound or pharmaceutical composition as described herein, or a pharmaceutically acceptable salt, ester, prodrug, solvate, hydrate or derivative thereof to a subject in need thereof. In certain embodiments, the cancer is lung cancer, pancreatic cancer, or skin cancer. In certain embodiments, the cancer is lung cancer. In other embodiments, the cancer is pancreatic cancer. In yet other embodiments, the cancer is skin cancer. In specific embodiments, the cancer is melanoma.

Embodiments of the invention also relate to a method of treating a MRGPRD dependent hyperproliferative disorder in a mammal that comprises administering to said mammal a therapeutically effective amount of a compound or pharmaceutical composition as described herein, or a pharmaceutically acceptable salt, ester, prodrug, solvate, hydrate or derivative thereof. In some embodiments, said method relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer.

In some embodiments, the invention relates to methods for treatment of a cancer related condition wherein the cancer related condition is lung cancer, pancreatic cancer, or skin cancer. In certain embodiments, the invention relates to methods for treatment of a cancer related condition wherein the cancer related condition is lung cancer. In other embodiments, the invention relates to methods for treatment of a cancer related condition wherein the cancer related condition is pancreatic cancer. In yet other embodiments, the invention relates to methods for treatment of a cancer related condition wherein the cancer related condition is skin cancer. In specific embodiments, the invention relates to methods for treatment of a cancer related condition wherein the cancer related condition is melanoma.

In certain particular embodiments, the invention relates to methods for treatment of MRGPRD dependent lung cancers, the methods comprise administering an effective amount of any of the above-described compound (or a pharmaceutical composition comprising the same) to a subject in need thereof. In certain embodiments the MRGPRD dependent lung cancer is a non-small cell lung carcinoma (NSCLC), for example adenocarcinoma, squamous-cell lung carcinoma or large-cell lung carcinoma. In other embodiments, the MRGPRD dependent lung cancer is a small cell lung carcinoma. Other MRGPRD dependent lung cancers treatable with the disclosed compounds include, but are not limited to, glandular tumors, carcinoid tumors and undifferentiated carcinomas.

Subjects that can be treated with compounds or pharmaceutical compositions of the invention, or pharmaceutically acceptable salt, isomer, hydrate, solvate or isotope of said compounds, according to the methods of this invention include, for example, subjects that have been diagnosed as having acute myeloid leukemia, acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myeloproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer.

Embodiments of the invention also relate to a method of treating a MRGPRD dependent hyperproliferative disorder in a mammal that comprises administering to said mammal a therapeutically effective amount of a compound or pharmaceutical composition as described herein, or a pharmaceutically acceptable salt, ester, prodrug, solvate, hydrate or derivative thereof. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)). In some embodiments subjects that are treated with the compounds or pharmaceutical compositions of the invention include subjects that have been diagnosed as having a non-cancerous hyperproliferative disorder such as benign hyperplasia of the skin (e.g., psoriasis), restenosis, or prostate (e.g., benign prostatic hypertrophy (BPH)).

As used herein, the phrase “MRGPRD dependent condition” means a condition where the activation, over sensitization, or desensitization of MRGPRD by a natural or synthetic ligand initiates, mediates, sustains, or augments a pathological condition.

A method of treating a subject having a pathological condition is provided, the method comprising of the administration to the subject a pharmaceutically effective amount of a compound having structure (I) or a pharmaceutically acceptable salt, isomer, hydrate, solvate or isotope thereof, or a pharmaceutical composition thereof.

As mentioned above, the invention relates to modulators of MRGPRD, to products containing the same, as well as to methods of their use and preparation. This receptor mediates disorders including dry eye syndrome/keratoconjunctivitis sicca and related conditions, chronic itch (e.g., pruritus), inflammation disorders, autoimmunity, skin disorders, cardiovascular disease, renal disease, cognitive impairment due to neurodegenerative diseases, age-induced cognitive impairment, vascular cognitive impairment, post-stroke cognitive impairment, and psychiatric disorders.

### Definitions

As used herein, the following terms have the meaning defined below, unless the context indicates otherwise.

“Modulating” MRGPRD means that the compound interacts with the MRGPRD in a manner such that it functions as an agonist to the receptor. In one embodiment, such modulation is partially or fully selective against other MRGPRs, such as MRGPRX1, X2, X3, X4, E,F, and/or G.

The term “agonism” is used herein to encompass compounds that interact in some way with a receptor and thereby function as an agonist, either by binding to the receptor at the binding site of its natural ligand or at locations other than the binding site. Thus, the phrase “MRGPRD agonism” is used herein to encompass compounds that interact in some way with MRGPRD and thereby function as an agonist, either by binding to the GPCR receptor at the binding site of its natural ligand, or at a location other than the binding site (i.e., allosteric binding).

Conversely, the term “antagonism” is used herein to encompass compounds that interact in some way with a receptor and thereby function as an antagonist, either by binding to the receptor at the binding site of its natural ligand or at locations other than the binding site. Thus, the phrase “ MRGPRD antagonism” is used herein to encompass compounds that interact in some way with the MRGPRD and thereby function as an antagonist, either by binding to the GPCR at the binding site of its natural ligand, or at a location other than the binding site (i.e., allosteric binding).

A partial agonist is a compound that binds to and activates a receptor, but with reduced efficacy compared to a full agonist. In the presence of a full agonist, a partial agonist behaves as an effective competitive antagonist. An inverse agonist is a compound that binds to a receptor and induces an opposing pharmacological response to that of an agonist. An allosteric modulator is a compound that binds at a location distinct from the orthosteric site, or the site of action of the primary ligand, and exerts an indirect effect by influencing binding or efficacy of the primary ligand. Pure allostery exerts no effect on a protein in the absence of a primary ligand that either activates or deactivates a receptor.

“MRGPR” refers to one or more of the Mas-related G protein coupled receptors, which are a group of orphan receptors with limited expression in very specialized tissues (e.g., in mast cells and dorsal root ganglia) and barrier tissues. There are eight related receptors in this class expressed in humans, only 4 of which have readily identifiable orthologs in other species (i.e., MRGPRD, E, F and G). Some of the other four receptors (MRGPRX1, X2, X3 and X4) have counterparts in higher species including dogs and horses, but they do not have a single corresponding ortholog in rodents.

“MRGPRD,” also referred to as “MRGD,” or,” TGR⁷″, or “MAS related GPR family member D” refers to a member of the MRGPR family.

“Effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an” and “the” include plural references unless the context clearly dictates otherwise.

In one embodiment, compounds are provided having the structure of Formula (I):

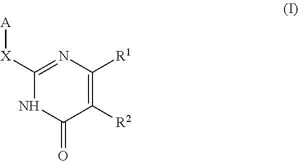

or a pharmaceutically acceptable isomer, racemate, hydrate, solvate, isotope, or salt thereof, wherein
A is,

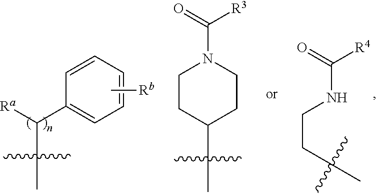

X is NH or O;
R¹ is H or alkyl;
R² is H or alkyl;
or R¹ and R² taken together with the atoms to which they are attached form carbocycle;
n is 0 or 1;
R^{a} is H or alkyl;
R^{b} is H, CN, alkyl, alkoxy or NR^{c}R^{d};
R^{e} and R^{d} are the same or different and independently H or alkyl;
R³ is —[(CH₂)ₘCR^{e}R^{f})]ₚ—R^{g};
R⁴ is —(CH₂)ₘCR^{e}R^{f})—R^{g};
m is 0 or 1;
p is 0 or1;
R^{e} and R^{f} are the same or different and independently H or alkyl, or R^{e} and R^{e} taken together with the atom to which they are attached form cycloalkyl; and
R⁸ is unsubstituted phenyl or phenyl substituted with R^{b}.

As used herein, “alkyl” means a saturated or unsaturated straight chain or branched alkyl group having from 1 to 8 carbon atoms, in some embodiments from 1 to 6 carbon atoms, in some embodiments from 1 to 4 carbon atoms, and in some embodiments from 1 to 3 carbon atoms. Examples of alkyl groups include, but are not limited to, methyl, ethyl, n-propyl, isopropyl, n-butyl, sec-butyl, isobutyl, or tert-butyl. Examples of saturated straight chain alkyl groups include, but are not limited to, methyl, ethyl, n-propyl, n-butyl, n-pentyl-, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. An unsaturated alkyl includes alkenyl and alkynyl as defined below. Specific examples of alkyl groups include,

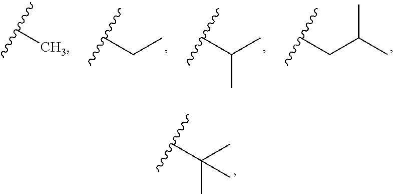

and the like.

As used herein, “alkenyl” means a straight chain or branched alkenyl group having from 2 to 8 carbon atoms, in some embodiments from 2 to 6 carbon atoms, in some embodiments from 2 to 4 carbon atoms, and in some embodiments from 2 to 3 carbon atoms. Alkenyl groups are unsaturated hydrocarbons that contain at least one carbon-carbon double bond. Examples of lower alkenyl groups include, but are not limited to, vinyl, propenyl, butenyl, pentenyl, and hexenyl.

As used herein, “alkynyl” means a straight chain or branched alkynyl group having from 2 to 8 carbon atoms, in some embodiments from 2 to 6 carbon atoms, in some embodiments from 2 to 4 carbon atoms, and in some embodiments from 2 to 3 carbon atoms. Alkynyl groups are unsaturated hydrocarbons that contain at least one carbon-carbon triple bond. Examples of alkynyl groups include, but are not limited to, ethynyl, propynyl, butynyl, pentynyl, and hexynyl.
“Halo” or “halogen” refers to fluorine, chlorine, bromine, and iodine.
“Hydroxy” or “hydroxyl” refers to —OH.
“Cyano” refers to —CN.
“Amino” refers to —NH₂.
“Aminyl” refers to —NHalkyl or N(alkyl)₂, wherein alkyl is as defined above. Examples of aminyl include, but are not limited to

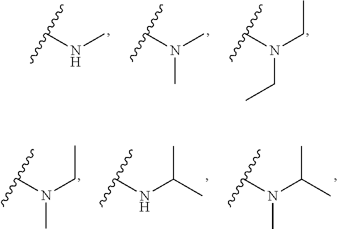

and the like.

“Haloalkyl” refers to alkyl as defined above with one or more hydrogen atoms replaced with halogen. Examples of lower haloalkyl groups include, but are not limited to, —CF₃, —CHF₂, and the like.

“Alkoxy” refers to alkyl as defined above joined by way of an oxygen atom (i.e., —O-alkyl). Examples of alkoxy groups include, but are not limited to, methoxy, ethoxy, n-propoxy, n-butoxy, isopropoxy, sec-butoxy, tert-butoxy, and the like. Examples of alkoxyalkyl groups include, but are not limited to,

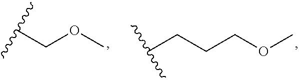

and the like.

“Haloalkoxy” refers to haloalkyl as defined above joined by way of an oxygen atom (i.e., —O-haloalkyl). Examples of lower haloalkoxy groups include, but are not limited to, —OCF₃, and the like.

“Cycloalkyl” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring do not give rise to aromaticity.

Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

“Aryl” groups are cyclic aromatic hydrocarbons that do not contain heteroatoms. Representative aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include fused rings wherein at least one ring, but not necessarily all rings, are aromatic, such as fused aromatic-aliphatic ring systems (e.g., indanyl, tetrahydronaphthyl, and the like). In one embodiment, aryl is phenyl or naphthyl, and in another embodiment aryl is phenyl.

“Carbocycle” refers to alkyl groups forming a ring structure, which can be substituted or unsubstituted, wherein the ring is either completely saturated, partially unsaturated, or fully unsaturated, wherein if there is unsaturation, the conjugation of the pi-electrons in the ring may give rise to aromaticity. In one embodiment, carbocycle includes cycloalkyl as defined above. In another embodiment, carbocycle includes aryl as defined above.

“Heterocycle” refers to aromatic and non-aromatic ring moieties containing 3 or more ring members, of which one or more is a heteroatom such as, but not limited to, N, O, S, or P. In some embodiments, heterocyclyl include 3 to 20 ring members, whereas other such groups have 3 to 15 ring members. At least one ring contains a heteroatom, but every ring in a polycyclic system need not contain a heteroatom. For example, a dioxolanyl ring and a benzdioxolanyl ring system (methylenedioxyphenyl ring system) are both heterocyclyl groups within the meaning herein.

Heterocyclyl groups also include fused ring species including those having fused aromatic and non-aromatic groups. A heterocyclyl group also includes polycyclic ring systems containing a heteroatom such as, but not limited to, quinuclidyl, and also includes heterocyclyl groups that have substituents, including but not limited to alkyl, halo, amino, hydroxy, cyano, carboxy, nitro, thio, or alkoxy groups, bonded to one of the ring members. A heterocyclyl group as defined herein can be a heteroaryl group or a partially or completely saturated cyclic group including at least one ring heteroatom. Heterocyclyl groups include, but are not limited to, pyrrolidinyl, furanyl, tetrahydrofuranyl, dioxolanyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

“Heteroaryl” refers to aromatic ring moieties containing 5 or more ring members, of which, one or more is a heteroatom such as, but not limited to, N, O, and S. Heteroaryl groups include, but are not limited to, groups such as pyrrolyl, pyrazolyl, pyridinyl, pyridazinyl, pyrimidyl, pyrazyl, pyrazinyl, pyrimidinyl, thienyl, triazolyl, tetrazolyl, triazinyl, thiazolyl, thiophenyl, oxazolyl, isoxazolyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl, and 2,3-dihydro indolyl.

The compounds of the disclosure (i.e., compounds having the structure of Formula (I), (II), (IIa), (III), (IIIa), (IV) or (IVa), and embodiments thereof), or their pharmaceutically acceptable salts may contain one or more centers of geometric asymmetry and may thus give rise to enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)- isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also included.

“Isomer” is used herein to encompass all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the invention. The isomers resulting from the presence of a chiral center comprise a pair of non-superimposable isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least 80%, at least 82%, at least 84%, at least 86%, or at least 88% pure by weight. In other embodiments, the isolated isomer is at least 90% pure. In another embodiment, the isolated isomer is at least 95% pure, at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(+)” because it is not optically active (i.e., will not rotate plane-polarized light in either direction since its constituent enantiomers cancel each other out). All compounds with an asterisk (*) adjacent to a tertiary or quaternary carbon are optically active isomers, which may be purified from the respective racemate and/or synthesized by appropriate chiral synthesis.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound as described herein includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine 19 is longest-lived. Thus, an isotope of a compound having the structure of Formula (I), (II), (IIa), (III), (IIIa), (IV) or (IVa), includes, but not limited to, compounds of Formula (I), (II), (IIa), (III), (IIIa), (IV) or (IVa), wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

Co-crystal forms of compounds having the structure of Formula (I), (II), (IIa), (III), (IIIa), (IV) or (IVa), are also included within the scope of this invention; namely, solids that are crystalline single phase materials composed of two or more different molecular and/or ionic compounds generally in a stoichiometric ratio which are neither solvates nor simple salts.

The term “pharmaceutically acceptable” refers an agent that has been approved for human consumption and is generally non-toxic. For example, the term “pharmaceutically acceptable salt” refers to non-toxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the invention include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, β-hydroxybutyric, salicylic,-galactaric, and galacturonic acid.

Although pharmaceutically unacceptable salts are not generally useful as medicaments, such salts may be useful, for example as intermediates in the synthesis of compounds having the structure (I), for example in their purification by recrystallization.

In one embodiment of Formula (I), A is

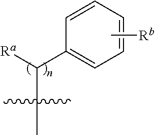

and compounds are provided having the structure of Formula (II):

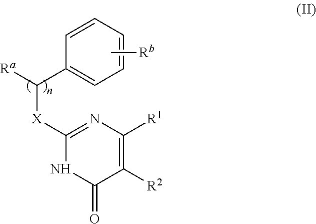

or a pharmaceutically acceptable isomer, racemate, hydrate, solvate, isotope, or salt thereof, wherein
X is NH or O;
R¹ is H or alkyl;
R² is H or alkyl;
or R¹ and R² taken together with the atoms to which they are attached form carbocycle;
n is 0 or 1;
R^{a} is H or alkyl;
R^{b} is H, CN, alkyl, alkoxy or NR^{c}R^{d}; and
R^{c} and R^{c} are the same or different and independently H or alkyl.

In one embodiment of Formula (II), X is NH, n is 1, R^{a} is H and compounds are provided having the structure of Formula (II-1):

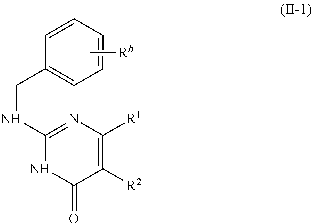

or a pharmaceutically acceptable isomer, racemate, hydrate, solvate, isotope, or salt thereof, wherein
R¹ is H or alkyl;
R² is H or alkyl;
or R¹ and R² taken together with the atoms to which they are attached form carbocycle;
R^{b} is H, CN, alkyl, alkoxy or NR^{b}R^{c}; and
R^{b} and R^{c} are the same or different and independently H or alkyl.

In one embodiment, provided are compounds having the structure of Formula (IIa),

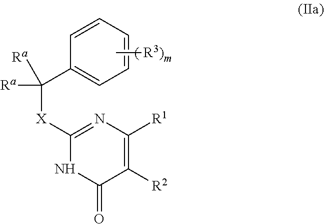

or a pharmaceutically acceptable isomer, racemate, hydrate, solvate, isotope, or salt thereof, wherein:
(A) X is NH;
   R¹ is methyl;
   R² is n-butyl;
   R³ is CN, alkyl, alkoxy, or aminyl; and
   either (1) each R^{a} is independently H or alkyl and m is 1, or (2) at least one R^{a} is alkyl and the other R^{a} is H or alkyl and m is 0 or 1; or
(B) X is O;
   R¹ is alkyl and R² is alkyl, or R¹ and R² join together to form

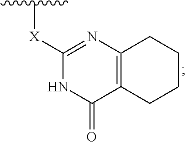

R³ is CN, alkyl, alkoxy, or aminyl;
each Ra is independently H or alkyl; and
m is 0 or 1.

In some embodiments, provided are compounds having the structure of Formula (IIa), wherein X is O and R¹ and R² are both alkyl. In some embodiments, provided are compounds having the structure of Formula (IIa), wherein each alkyl is independently methyl, ethyl, n-propyl, isopropyl, n-butyl, sec-butyl, isobutyl, or tert-butyl. In some embodiments, provided are compounds having the structure of Formula (IIa), wherein R¹ is methyl and R² is n-butyl.

In some embodiments, provided are compounds having the structure of Formula (IIa), wherein X is O and R¹ and R² join together to form

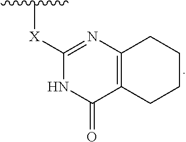

In some embodiments, provided are compounds having the structure of Formula (IIa), wherein X is NH.

In some embodiments, provided are compounds having the structure of Formula (IIa), wherein each R^{a} is independently H or alkyl and m is 1. In some embodiments, provided are compounds having the structure of Formula (IIa), wherein each R^{a} is H and m is 1. In some embodiments, provided are compounds having the structure of Formula (IIa), wherein each R^{a} is alkyl and m is 1.

In some embodiments, provided are compounds having the structure of Formula (IIa), wherein one R^{a} is H, the other Ra is alkyl, and m is 0. In some embodiments, provided are compounds having the structure of Formula (IIa), wherein one R^{a} is H, the other R^{a} is methyl, and m is 0. In some embodiments, provided are compounds having the structure of Formula (IIa), wherein each R^{a} is alkyl and m is 0.

In some embodiments, provided are compounds having the structure of Formula (IIa), wherein R³ is CN, alkyl, or alkoxy. In some embodiments, provided are compounds having the structure of Formula (IIa), wherein R³ is CN, alkoxy, or aminyl. In some embodiments, provided are compounds having the structure of Formula (IIa), wherein R³ is alkyl, alkoxy, or aminyl. In some embodiments, provided are compounds having the structure of Formula (IIa), wherein R³ is CN, alkyl, or aminyl.

In some embodiments, provided are compounds having the structure of Formula (IIa), wherein R³ is CN or alkyl. In some embodiments, provided are compounds having the structure of Formula (IIa), wherein R³ is CN or alkoxy. In some embodiments, provided are compounds having the structure of Formula (IIa), wherein R³ is CN or aminyl. In some embodiments, provided are compounds having the structure of Formula (IIa), wherein R³ is alkyl or alkoxy. In some embodiments, provided are compounds having the structure of Formula (IIa), wherein R³ is alkyl or aminyl. In some embodiments, provided are compounds having the structure of Formula (IIa), wherein R³ is alkoxy or aminyl.

In some embodiments, provided are compounds having the structure of Formula (IIa), wherein R³ is CN.

In some embodiments, provided are compounds having the structure of Formula (IIa), wherein R³ is alkyl. In some embodiments, provided are compounds having the structure of Formula (IIa), wherein R³ is methyl, ethyl, n-propyl, or isopropyl. In some embodiments, provided are compounds having the structure of Formula (IIa), wherein R³ is methyl.

In some embodiments, provided are compounds having the structure of Formula (IIa), wherein R³ is alkoxy.

In some embodiments, provided are compounds having the structure of Formula (IIa), wherein R³ is methoxy, ethoxy, n-propoxy, or isopropoxy. In some embodiments, provided are compounds having the structure of Formula (IIa), wherein R³ is methoxy.

In some embodiments, provided are compounds having the structure of Formula (IIa), wherein R³ is aminyl. In some embodiments, provided are compounds having the structure of Formula (IIa), wherein R³ is —N(CH₃)₂.

In one embodiment of Formula (I), A is

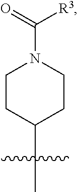

X is NH, and compounds are provided having the structure of Formula (III):

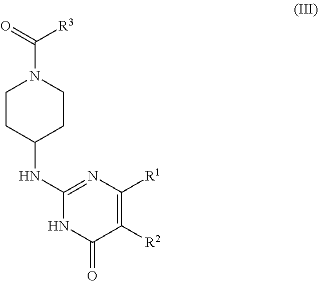

or a pharmaceutically acceptable isomer, racemate, hydrate, solvate, isotope, or salt thereof, wherein
R¹ is H or alkyl;
R² is H or alkyl;
or R¹ and R² taken together with the atoms to which they are attached form carbocycle;
R³ is —[(CH₂)ₘCR^{e}R^{f})]ₚ—R^{g};
m is 0 or 1;
p is 0 or 1;
R^{e} and R^{f} are the same or different and independently H or alkyl, or R^{e} and R^{e} taken together with the atom to which they are attached form cycloalkyl; and
R^{g} is unsubstituted phenyl or phenyl substituted with R^{b}.
R^{b} is H, CN, alkyl, alkoxy or NR^{c}R^{d}; and
R^{c} and R^{d} are the same or different and independently H or alkyl.

In some embodiments, provided are compounds having the structure of Formula (IIIa),

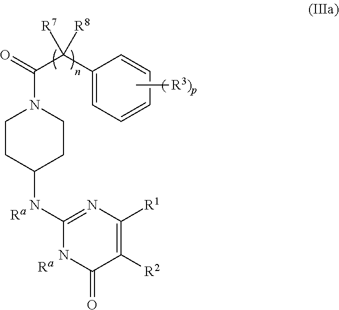

or a pharmaceutically acceptable isomer, racemate, hydrate, solvate, isotope, or salt thereof, wherein:
(A) R¹ is H and R² is H, halo, or alkyl; or R¹ and R² join together to form

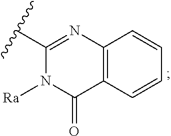

R³ is CN, alkyl, alkoxy, or aminyl;
   each R⁷ and R⁸ are independently H or alkyl, or R⁷ and R⁸ when attached to the same carbon join together to form a cycloalkyl ring;
   n is 0, 1, or 2; and
   p is 0 or 1; or
(B) R¹ and R² join together to form

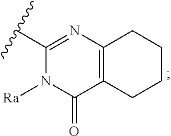

R³ is CN, alkyl, or alkoxy;
each R⁷ and R⁸ are independently H or alkyl;
n is 0, 1, or 2; and
p is 0 or 1.

In some embodiments, provided are compounds having the structure of Formula (IIIa), wherein both R¹ and R² are H.

In some embodiments, provided are compounds having the structure of Formula (IIIa), wherein R¹ is H and R² is halo.

In some embodiments, provided are compounds having the structure of Formula (IIIa), wherein R² is F, Cl, Br, or I.

In some embodiments, provided are compounds having the structure of Formula (IIIa), wherein R² is F.

In some embodiments, provided are compounds having the structure of Formula (IIIa), wherein R¹ is H and R² is alkyl. In some embodiments, provided are compounds having the structure of Formula (IIIa), wherein the alkyl is methyl, ethyl, n-propyl, or isopropyl. In some embodiments, provided are compounds having the structure of Formula (IIIa), wherein the alkyl is methyl.

In some embodiments, provided are compounds having the structure of Formula (IIIa), wherein R¹ and R² join together to form

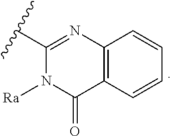

In some embodiments, provided are compounds having the structure of Formula (IIIa), wherein R¹ and R² join together to form

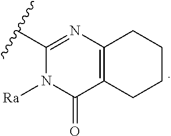

In some embodiments, provided are compounds having the structure of Formula (IIIa), wherein each R^{a} is H.

In some embodiments, provided are compounds having the structure of Formula (IIIa), wherein each R^{a} is alkyl.

In some embodiments, provided are compounds having the structure of Formula (IIIa), wherein one R^{a} is H and the other R^{a} is alkyl. In some embodiments, provided are compounds having the structure of Formula (IIIa), wherein the alkyl is methyl, ethyl, n-propyl, or isopropyl. In some embodiments, provided are compounds having the structure of Formula (IIIa), wherein the alkyl is methyl.

In some embodiments, provided are compounds having the structure of Formula (IIIa), wherein R³ is CN, alkyl, or alkoxy. In some embodiments, provided are compounds having the structure of Formula (IIIa), wherein R³ is CN, alkoxy, or aminyl. In some embodiments, provided are compounds having the structure of Formula (IIIa), wherein R³ is alkyl, alkoxy, or aminyl. In some embodiments, provided are compounds having the structure of Formula (IIIa), wherein R³ is CN, alkyl, or aminyl.

In some embodiments, provided are compounds having the structure of Formula (IIIa), wherein R³ is CN or alkyl. In some embodiments, provided are compounds having the structure of Formula (IIIa), wherein R³ is CN or alkoxy. In some embodiments, provided are compounds having the structure of Formula (IIIa), wherein R³ is CN or aminyl. In some embodiments, provided are compounds having the structure of Formula (IIIa), wherein R³ is alkyl or alkoxy. In some embodiments, provided are compounds having the structure of Formula (IIIa), wherein R³ is alkyl or aminyl. In some embodiments, provided are compounds having the structure of Formula (IIIa), wherein R³ is alkoxy or aminyl.

In some embodiments, provided are compounds having the structure of Formula (IIIa), wherein R³ is CN

In some embodiments, provided are compounds having the structure of Formula (IIIa), wherein R³ is alkyl. In some embodiments, provided are compounds having the structure of Formula (IIIa), wherein R³ is methyl, ethyl, n-propyl, or isopropyl. In some embodiments, provided are compounds having the structure of Formula (IIIa), wherein R³ is methyl.

In some embodiments, provided are compounds having the structure of Formula (IIIa), wherein R³ is alkoxy. In some embodiments, provided are compounds having the structure of Formula (IIIa), wherein R³ is methoxy, ethoxy, n-propoxy, or isopropoxy. In some embodiments, provided are compounds having the structure of Formula (IIIa), wherein R³ is methoxy.

In some embodiments, provided are compounds having the structure of Formula (IIIa), wherein R³ is aminyl. In some embodiments, provided are compounds having the structure of Formula (IIIa), wherein R³ is —N(CH₃)₂.

In some embodiments, provided are compounds having the structure of Formula (IIIa), wherein each R⁷ is H.

In some embodiments, provided are compounds having the structure of Formula (IIIa), wherein each R⁷ is alkyl.

In some embodiments, provided are compounds having the structure of Formula (IIIa), wherein each R⁸ is H.

In some embodiments, provided are compounds having the structure of Formula (IIIa), wherein each R⁸ is alkyl.

In some embodiments, provided are compounds having the structure of Formula (IIIa), wherein R⁷ and R⁸ are both H.

In some embodiments, provided are compounds having the structure of Formula (IIIa), wherein R⁷ and R⁸ are both alkyl. In some embodiments, provided are compounds having the structure of Formula (IIIa), wherein the alkyl is methyl, ethyl, n-propyl, or isopropyl. In some embodiments, provided are compounds having the structure of Formula (IIIa), wherein the alkyl is methyl.

In some embodiments, provided are compounds having the structure of Formula (IIIa), wherein R⁷ and R⁸ are attached to the same carbon and join together to form a cycloalkyl ring. In some embodiments, provided are compounds having the structure of Formula (IIIa), wherein the cycloalkyl ring is cyclopropyl.

In some embodiments, provided are compounds having the structure of Formula (IIIa), wherein n is 0.

In some embodiments, provided are compounds having the structure of Formula (IIIa), wherein n is 1.

In some embodiments, provided are compounds having the structure of Formula (IIIa), wherein n is 2.

In some embodiments, provided are compounds having the structure of Formula (IIIa), wherein p is 0.

In some embodiments, provided are compounds having the structure of Formula (IIIa), wherein p is 1.

In one embodiment of Formula (I), A is,

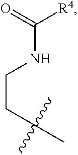

X is NH, and compounds are provided having the structure of Formula (IV):

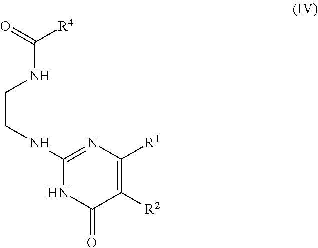

or a pharmaceutically acceptable isomer, racemate, hydrate, solvate, isotope, or salt thereof, wherein
R¹ is H or alkyl;
R² is H or alkyl;
or R¹ and R² taken together with the atoms to which they are attached form carbocycle saturated or unsaturated;
R⁴ is —(CH₂)ₘCR^{e}R^{f})—R^{g};
m is 0 or 1;
R^{e} and R^{f} are the same or different and independently H or alkyl, or R^{e} and R^{e} taken together with the atom to which they are attached form cycloalkyl; and
R⁸ is unsubstituted phenyl or phenyl substituted with R^{b}.
R^{b} is H, CN, alkyl, alkoxy or NR^{c}R^{d}; and
R^{c} and R^{d} are the same or different and independently H or alkyl.

In some embodiments, provided are compounds having the structure of Formula (IVa),

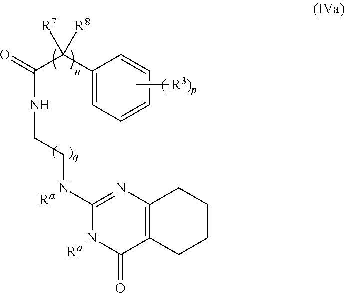

or a pharmaceutically acceptable isomer, racemate, hydrate, solvate, isotope, or salt thereof, wherein:
   R³ is CN, alkyl, alkoxy, or aminyl;
   each R⁷ is independently H or alkyl, each R⁸ is independently H or alkyl, or R⁷ and R⁸ are attached to the same carbon and join together to form a cycloalkyl ring;
   each R^{a} is independently H or alkyl;
   n is 1-2;
   p is 0 or 1; and
   q is 1-2.

In some embodiments, provided are compounds having the structure of Formula (IVa), wherein R³ is CN, alkyl, or alkoxy. In some embodiments, provided are compounds having the structure of Formula (IVa), wherein R³ is CN, alkoxy, or aminyl. In some embodiments, provided are compounds having the structure of Formula (IVa), wherein R³ is alkyl, alkoxy, or aminyl. In some embodiments, provided are compounds having the structure of Formula (IVa), wherein R³ is CN, alkyl, or aminyl.

In some embodiments, provided are compounds having the structure of Formula (IVa), wherein R³ is CN or alkyl. In some embodiments, provided are compounds having the structure of Formula (IVa), wherein R³ is CN or alkoxy. In some embodiments, provided are compounds having the structure of Formula (IVa), wherein R³ is CN or aminyl. In some embodiments, provided are compounds having the structure of Formula (IVa), wherein R³ is alkyl or alkoxy. In some embodiments, provided are compounds having the structure of Formula (IVa), wherein R³ is alkyl or aminyl. In some embodiments, provided are compounds having the structure of Formula (IVa), wherein R³ is alkoxy or aminyl.

In some embodiments, provided are compounds having the structure of Formula (IVa), wherein R³ is CN.

In some embodiments, provided are compounds having the structure of Formula (IVa), wherein R³ is alkyl. In some embodiments, provided are compounds having the structure of Formula (IVa), wherein R³ is methyl, ethyl, n-propyl, or isopropyl. In some embodiments, provided are compounds having the structure of Formula (IVa), wherein R³ is methyl.

In some embodiments, provided are compounds having the structure of Formula (IVa), wherein R³ is alkoxy. In some embodiments, provided are compounds having the structure of Formula (IVa), wherein R³ is methoxy, ethoxy, n-propoxy, or isopropoxy. In some embodiments, provided are compounds having the structure of Formula (IVa), wherein R³ is methoxy.

In some embodiments, provided are compounds having the structure of Formula (IVa), wherein R³ is aminyl. In some embodiments, provided are compounds having the structure of Formula (IVa), wherein R³ is —N(CH₃)₂.

In some embodiments, provided are compounds having the structure of Formula (IVa), wherein each R⁷ is H.

In some embodiments, provided are compounds having the structure of Formula (IVa), wherein each R⁷ is alkyl.

In some embodiments, provided are compounds having the structure of Formula (IVa), wherein each R⁸ is H.

In some embodiments, provided are compounds having the structure of Formula (IVa), wherein each R⁸ is alkyl.

In some embodiments, provided are compounds having the structure of Formula (IVa), wherein R⁷ and R⁸ are both H.

In some embodiments, provided are compounds having the structure of Formula (IVa), wherein R⁷ and R⁸ are both alkyl. In some embodiments, provided are compounds having the structure of Formula (IVa), wherein the alkyl is methyl, ethyl, n-propyl, or isopropyl. In some embodiments, provided are compounds having the structure of Formula (IVa), wherein the alkyl is methyl.

In some embodiments, provided are compounds having the structure of Formula (IVa), wherein R⁷ and R⁸ are attached to the same carbon and join together to form a cycloalkyl ring. In some embodiments, provided are compounds having the structure of Formula (IVa), wherein the cycloalkyl ring is cyclopropyl.

In some embodiments, provided are compounds having the structure of Formula (IVa), wherein each R^{a} is H.

In some embodiments, provided are compounds having the structure of Formula (IVa), wherein each R^{a} is alkyl. In some embodiments, provided are compounds having the structure of Formula (IVa), wherein one R^{a} is H and the other R^{a} is alkyl. In some embodiments, provided are compounds having the structure of Formula (IVa), wherein the alkyl is methyl, ethyl, n-propyl, or isopropyl. In some embodiments, provided are compounds having the structure of Formula (IVa), wherein the alkyl is methyl.

In some embodiments, provided are compounds having the structure of Formula (IVa), wherein n is 0. In some embodiments, provided are compounds having the structure of Formula (IVa), wherein n is 1. In some embodiments, provided are compounds having the structure of Formula (IVa), wherein n is 2.

In some embodiments, provided are compounds having the structure of Formula (IVa), wherein p is 0. In some embodiments, provided are compounds having the structure of Formula (IVa), wherein p is 1.

In some embodiments, provided are compounds having the structure of Formula (IVa), wherein q is 1. In some embodiments, provided are compounds having the structure of Formula (IVa), wherein q is 2.

Representative compounds of Formulas (I), (II), (IIa), (III), (IIIa) (IV) and (IVa) include the compounds listed in the Table below, as well as pharmaceutically acceptable isomers, racemates, hydrates, solvates, isotopes, and salts thereof. To this end, representative compounds are identified herein by their respective “Compound Number”, which is sometimes abbreviated as “Compound No.”, “Cpd. No.” or “No.”

TABLE A
Representative Compounds

| Cpd. No. | Structure |
|---|---|
| 1-1 | 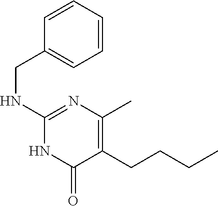 [SMILES: CCCCc1c(C)nc(NCc2ccccc2)[nH]c1=O] |
| 1-2 | 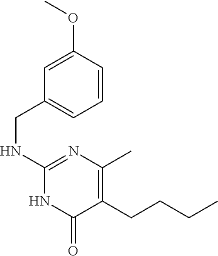 [SMILES: CCCCc1c(C)nc(NCc2cccc(OC)c2)[nH]c1=O] |
| 1-3 | 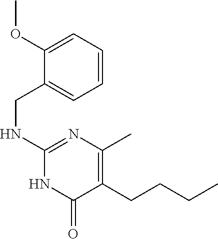 [SMILES: CCCCc1c(C)nc(NCc2ccccc2OC)[nH]c1=O] |
| 1-4 | 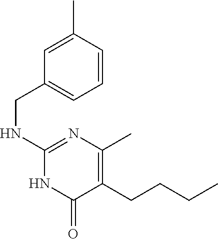 [SMILES: CCCCc1c(C)nc(NCc2cccc(C)c2)[nH]c1=O] |
| 1-5 | 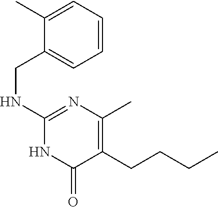 [SMILES: CCCCc1c(C)nc(NCc2ccccc2C)[nH]c1=O] |
| 1-6 | 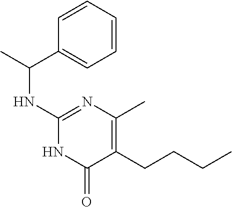 [SMILES: CCCCc1c(C)nc(NC(C)c2ccccc2)[nH]c1=O] |
| 1-7 | 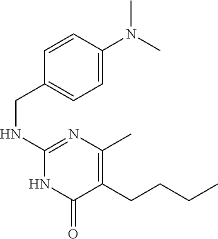 [SMILES: CCCCc1c(C)nc(NCc2ccc(N(C)C)cc2)[nH]c1=O] |
| 1-8 | 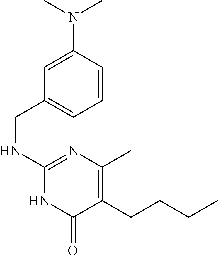 [SMILES: CCCCc1c(C)nc(NCc2cccc(N(C)C)c2)[nH]c1=O] |
| 1-9 | 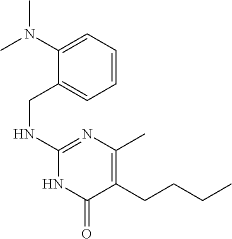 [SMILES: CCCCc1c(C)nc(NCc2ccccc2N(C)C)[nH]c1=O] |
| 1-10 | 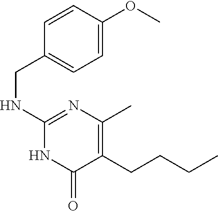 [SMILES: CCCCc1c(C)nc(NCc2ccc(OC)cc2)[nH]c1=O] |
| 1-11 | 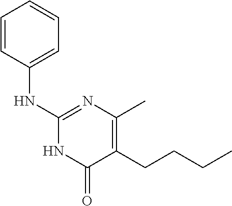 [SMILES: CCCCc1c(C)nc(Nc2ccccc2)[nH]c1=O] |
| 2-1 | 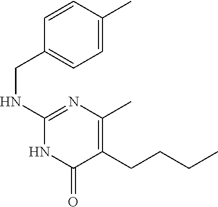 [SMILES: CCCCc1c(C)nc(NCc2ccc(C)cc2)[nH]c1=O] |
| 2-2 | 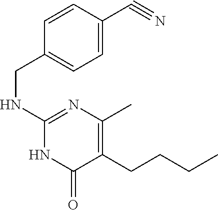 [SMILES: CCCCc1c(C)nc(NCc2ccc(C#N)cc2)[nH]c1=O] |
| 2-3 | 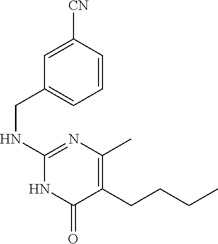 [SMILES: CCCCc1c(C)nc(NCc2cccc(C#N)c2)[nH]c1=O] |
| 3-1 | 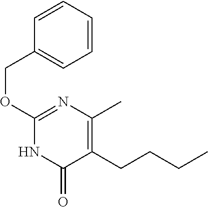 [SMILES: CCCCc1c(C)nc(OCc2ccccc2)[nH]c1=O] |
| 4-1 | 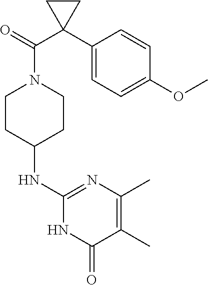 [SMILES: COc1ccc(C2(C(=O)N3CCC(Nc4nc(C)c(C)c(=O)[nH]4)CC3)CC2)cc1] |
| 4-2 | 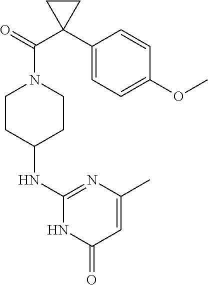 [SMILES: COc1ccc(C2(C(=O)N3CCC(Nc4nc(C)cc(=O)[nH]4)CC3)CC2)cc1] |
| 4-3 | 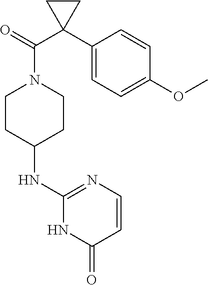 [SMILES: COc1ccc(C2(C(=O)N3CCC(Nc4nccc(=O)[nH]4)CC3)CC2)cc1] |
| 4-4 | 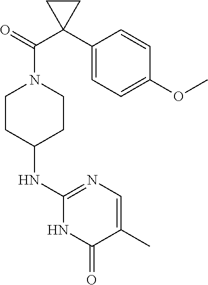 [SMILES: COc1ccc(C2(C(=O)N3CCC(Nc4ncc(C)c(=O)[nH]4)CC3)CC2)cc1] |
| 4-5 | 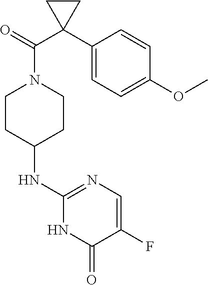 [SMILES: COc1ccc(C2(C(=O)N3CCC(Nc4ncc(F)c(=O)[nH]4)CC3)CC2)cc1] |
| 5-1 | 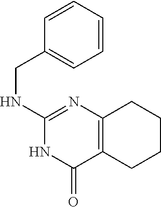 [SMILES: O=c1[nH]c(NCc2ccccc2)nc2c1CCCC2] |
| 6-1 | 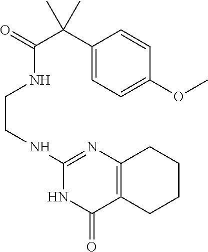 [SMILES: COc1ccc(C(C)(C)C(=O)NCCNc2nc3c(c(=O)[nH]2)CCCC3)cc1] |
| 7-1 | 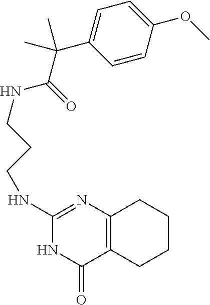 [SMILES: COc1ccc(C(C)(C)C(=O)NCCCNc2nc3c(c(=O)[nH]2)CCCC3)cc1] |
| 8-1 | 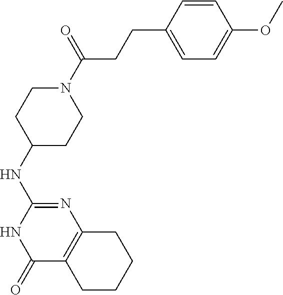 [SMILES: COc1ccc(CCC(=O)N2CCC(Nc3nc4c(c(=O)[nH]3)CCCC4)CC2)cc1] |
| 8-2 | 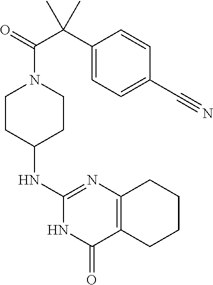 [SMILES: CC(C)(C(=O)N1CCC(Nc2nc3c(c(=O)[nH]2)CCCC3)CC1)c1ccc(C#N)cc1] |
| 8-3 | 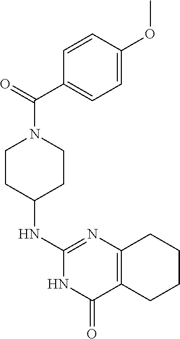 [SMILES: COc1ccc(C(=O)N2CCC(Nc3nc4c(c(=O)[nH]3)CCCC4)CC2)cc1] |
| 8-4 | 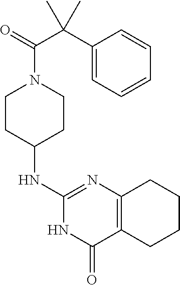 [SMILES: CC(C)(C(=O)N1CCC(Nc2nc3c(c(=O)[nH]2)CCCC3)CC1)c1ccccc1] |
| 8-5 | 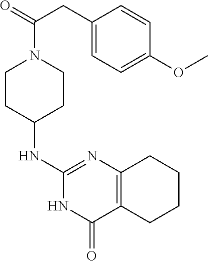 [SMILES: COc1ccc(CC(=O)N2CCC(Nc3nc4c(c(=O)[nH]3)CCCC4)CC2)cc1] |
| 8-6 | 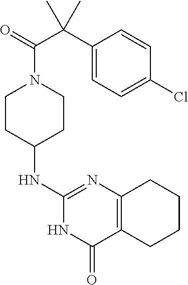 [SMILES: CC(C)(C(=O)N1CCC(Nc2nc3c(c(=O)[nH]2)CCCC3)CC1)c1ccc(Cl)cc1] |
| 8-7 | 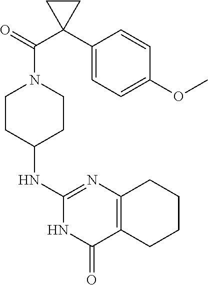 [SMILES: COc1ccc(C2(C(=O)N3CCC(Nc4nc5c(c(=O)[nH]4)CCCC5)CC3)CC2)cc1] |
| 9-1 | 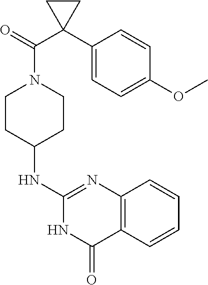 [SMILES: COc1ccc(C2(C(=O)N3CCC(Nc4nc5ccccc5c(=O)[nH]4)CC3)CC2)cc1] |

In one embodiment, provided is a compound having one of the following structures:

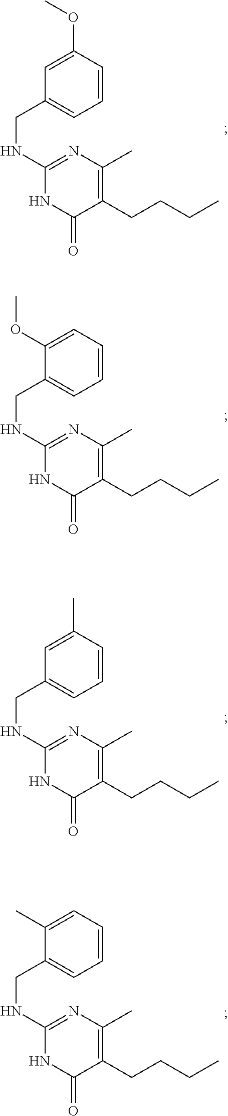 [SMILES: CCCCc1c(C)nc(NCc2cccc(OC)c2)[nH]c1=O ; CCCCc1c(C)nc(NCc2ccccc2OC)[nH]c1=O ; CCCCc1c(C)nc(NCc2cccc(C)c2)[nH]c1=O ; CCCCc1c(C)nc(NCc2ccccc2C)[nH]c1=O]

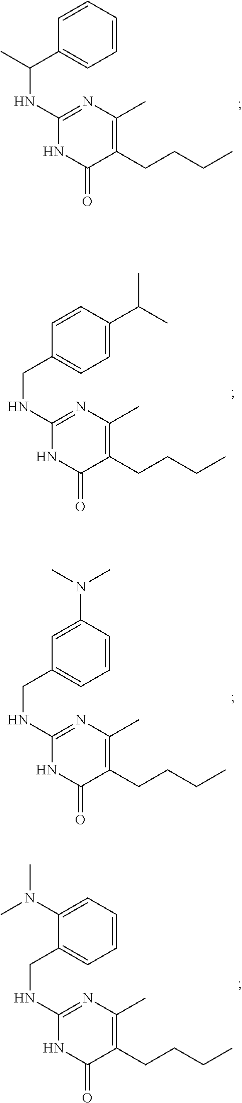

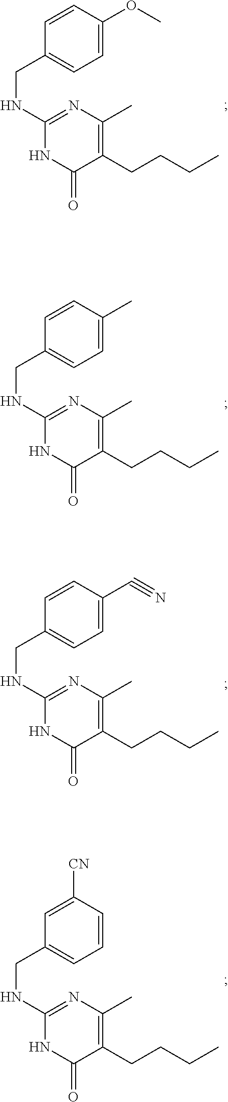 [SMILES: CCCCc1c(C)nc(NCc2ccc(OC)cc2)[nH]c1=O ; CCCCc1c(C)nc(NCc2ccc(C)cc2)[nH]c1=O ; CCCCc1c(C)nc(NCc2ccc(C#N)cc2)[nH]c1=O ; CCCCc1c(C)nc(NCc2cccc(C#N)c2)[nH]c1=O]

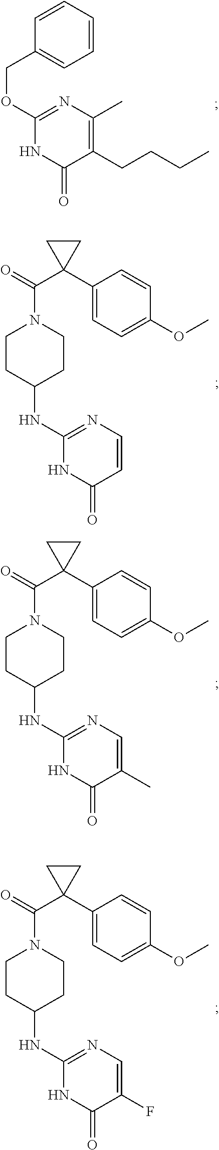

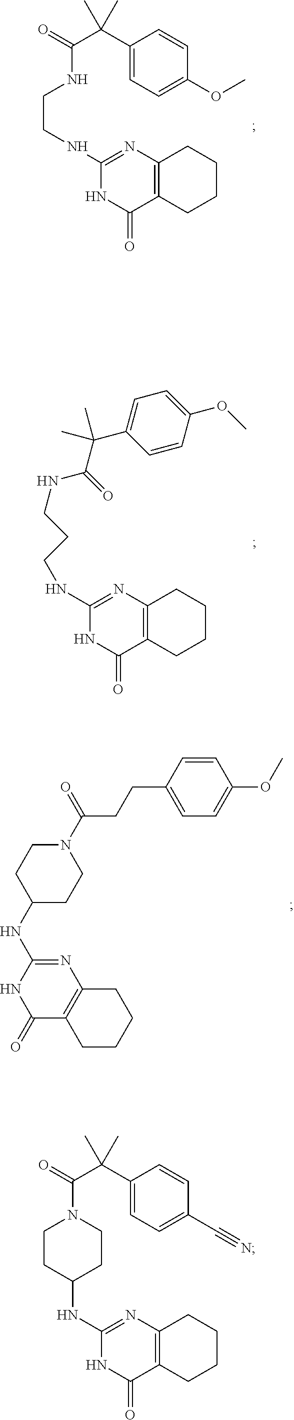 [SMILES: COc1ccc(C(C)(C)C(=O)NCCNc2nc3c(c(=O)[nH]2)CCCC3)cc1 ; COc1ccc(C(C)(C)C(=O)NCCCNc2nc3c(c(=O)[nH]2)CCCC3)cc1 ; COc1ccc(CCC(=O)N2CCC(Nc3nc4c(c(=O)[nH]3)CCCC4)CC2)cc1 ; CC(C)(C(=O)N1CCC(Nc2nc3c(c(=O)[nH]2)CCCC3)CC1)c1ccc(C#N)cc1]

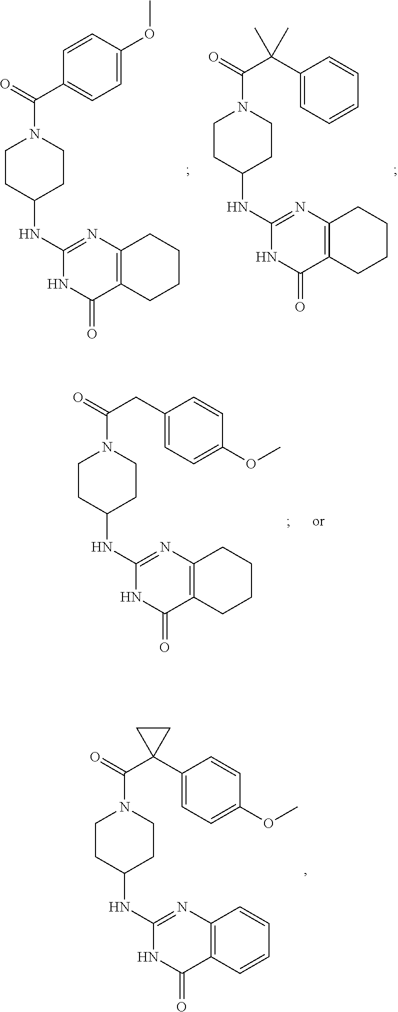 [SMILES: COc1ccc(C(=O)N2CCC(Nc3nc4c(c(=O)[nH]3)CCCC4)CC2)cc1 ; CC(C)(C(=O)N1CCC(Nc2nc3c(c(=O)[nH]2)CCCC3)CC1)c1ccccc1 ; COc1ccc(CC(=O)N2CCC(Nc3nc4c(c(=O)[nH]3)CCCC4)CC2)cc1 ; COc1ccc(C2(C(=O)N3CCC(Nc4nc5ccccc5c(=O)[nH]4)CC3)CC2)cc1]

In certain embodiments, the invention provides a pharmaceutical composition comprising a compound of the invention together with at least one pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethyl cellulose, and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal, or parenteral, e.g., rectal, depot, subcutaneous, intravenous, intraurethral, intramuscular, intranasal, ophthalmic solution, or an ointment, the oral route being preferred.

For parenteral administration, the carrier will typically comprise sterile water, although other ingredients that aid solubility or serve as preservatives can also be included. Furthermore, injectable suspensions can also be prepared, in which case appropriate liquid carriers, suspending agents, and the like can be employed.

For topical administration, the compounds of the present invention can be formulated using bland, moisturizing bases such as ointments or creams.

If a solid carrier is used for oral administration, the preparation can be tableted, placed in a hard gelatin capsule in powder or pellet form or it can be in the form of a troche or lozenge. If a liquid carrier is used, the preparation can be in the form of a syrup, emulsion, soft gelatin capsule, or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent. Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di-, or tri-glycerides.

For injection, the formulation can also be a powder suitable for reconstitution with an appropriate solution as described above. Examples of these include, but are not limited to, freeze dried, rotary dried, or spray dried powders, amorphous powders, granules, precipitates, or particulates. For injection, the formulations can optionally contain stabilizers, pH modifiers, surfactants, bioavailability modifiers, and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions of the present invention include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

Compositions of the present invention also include, for example, spray dry dispersions (e.g., a single-phase, amorphous molecular dispersion of a compound as described herein in a polymer matrix).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Dosage forms can be administered once a day, or more than once a day, such as twice or thrice daily. Alternatively, dosage forms can be administered less frequently than daily, such as every other day, or weekly, if found to be advisable by a prescribing physician. Dosing regimens include, for example, dose titration to the extent necessary or useful for the indication to be treated, thus allowing the patient's body to adapt to the treatment and/or to minimize or avoid unwanted side effects associated with the treatment. Other dosage forms include delayed or controlled-release forms. Suitable dosage regimens and/or forms include those set out, for example, in the latest edition of the Physicians' Desk Reference, incorporated herein by reference.

When used to prevent the onset of a malcondition, the compounds provided herein will be administered to a subject at risk for developing the same, typically on the advice and under the supervision of a physician, at the dosage levels described above. Subjects at risk for developing a particular malcondition generally include those that have a family history of the same, or those who have been identified by genetic testing or screening to be particularly susceptible to developing the malcondition.

Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

In another embodiment, there are provided methods of making a composition of a compound described herein including formulating a compound of the invention with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation.

As used herein, the phrase “MRGPRD or MRGPRD ortholog dependent condition” means a condition where the activation, over sensitization, or desensitization of MRGPRD or its ortholog by a natural or synthetic ligand initiates, mediates, sustains, or augments a pathological condition

In some embodiments, the MRGPRD dependent condition is a pain associated condition, an itch associated condition, an inflammatory condition, an ocular associated condition, a cardiovascular and renal disease associated condition, an inflammatory or autoimmune disorder, or a cognitive impairment associate condition.

As used herein, the phrase “pain associated condition” means any pain due to a medical condition. Thus, in one embodiment, the method of present invention is provided to treat a pain associated condition, such as Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Constipation, Corneal Neuropathic Pain, Crohn's Disease, Degenerative Disc Disease, Dental Pain, Dercum's Disease, Dermatomyositis, Diabetic Peripheral Neuropathy (DPN), Dry Eye Syndrome, Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Inflammatory bowel disease (IBD), Intercostal Neuralgia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, Ocular Itch, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rheumatica, Polymyositis, Porphyria, Post Herniorraphy Pain Syndrome, Post Mastectomy, Postoperative Pain, Pain Syndrome, Post Stroke Pain, Post Thorocotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, Sarcoidosi, Scheuemann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain or Vulvodynia.

As used herein, the phrase “itch associated condition” means pruritus (including acute and chronic pruritus) associated with any condition. The itch sensation can originate, e.g., from the peripheral nervous system (e.g., dermal or neuropathic itch) or from the central nervous system (e.g., neuropathic, neurogenic or psychogenic itch). Thus, in one embodiment, the method of present invention is provided to treat an itch associated condition, such as chronic itch; contact dermatitis; Allergic blepharitis; Anaphylaxis;

Anaphylactoid drug reactions; Anaphylactic shock; Anemia; Atopic dermatitis; Bullous pemphigoid; Candidiasis; Chicken pox; end-stage renal failure; hemodialysis; Cholestatic pruritis; Chronic urticaria; Contact dermatitis, Atopic Dermatitis; Dermatitis herpetiformis; Diabetes; Drug allergy, Dry Eye Syndrome; Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; lodinated contrast dye allergy; Iron deficiency anemia; Kidney disease; Leukemia, Porphyrias; Lymphoma; Mast cell activation syndrome; Malignancy; Mastocystosis; Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudo-allergic reactions; Pseudorabies; Psoriasis; Rectal prolapse; Sarcoidosis granulomas; Scabies; Schistosomiasis; Scleroderma; Severe stress; Stasia dermatitis; Swimmer's itch; Thyroid disease; Tinea cruris; Uremic Pruritus; Rosacea; Cutaneous amyloidosis; Scleroderma; Acne; wound healing; burn healing; ocular itch; and Urticaria. In a specific embodiment, the method of present invention is provided to treat an itch associated condition, such as urticaria, pruritus, atopic dermatitis, dry skin, psoriasis, contact dermatitis, or eczema.

As used herein, the term “administration” refers to providing a compound, or a pharmaceutical composition comprising the compound as described herein. The compound or composition can be administered by another person to the subject or it can be self-administered by the subject. Non-limiting examples of routes of administration are oral, parenteral (e.g., intravenous), or topical.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal, such as a human, dog or horse). A subject to be treated according to the methods described herein may be one who has been diagnosed with a MRGPRD dependent condition or MRGPRD ortholog dependent condition, such as a pain associated condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition. The term “patient” may be used interchangeably with the term “subject.” A subject may refer to an adult or pediatric subject.

The Federal Food, Drug, and Cosmetic Act defines “pediatric” as a subject aged 21 or younger at the time of their diagnosis or treatment. Pediatric subpopulations are further characterized as: (i) neonates -from birth through the first 28 days of life; (ii) infants-from 29 days to less than 2 years; (iii) children-2 years to less than 12 years; and (iv) adolescents-aged 12 through 21. Despite the definition, depending on the susceptible patient population and clinical trial evaluation, an approved regulatory label may include phrasing that specifically modifies the range of a pediatric population, such as, for example, pediatric patients up to 22 years of age.

In another embodiment, the method of treating a subject having a MRGPRD dependent condition (e.g., a pain associated conditions) described herein further comprises administering to the subject a pharmaceutically effective amount of a second therapeutic agent. In another embodiment, a method of treating a subject having a pain associated condition is provided, the method comprising administering to the subject a pharmaceutically effective amount of a compound having structure (I) or a pharmaceutically acceptable salt, isomer, hydrate, solvate or isotope thereof, or a pharmaceutical composition thereof.

In one embodiment, the malcondition for which modulation of MRGPRD is medically indicated is a pain associated condition, including: Acute Pain, Advanced Prostate Cancer, AIDS-Related Pain, Ankylosing Spondylitis, Arachnoiditis, Arthritis, Arthrofibrosis, Ataxic Cerebral Palsy, Autoimmune Atrophic Gastritis, Avascular Necrosis, Back Pain, Behcet's Disease (Syndrome), Burning Mouth Syndrome, Bursitis, Cancer Pain, Carpal Tunnel, Cauda Equina Syndrome, Central Pain Syndrome, Cerebral Palsy, Cervical Stenosis, Charcot-Marie-Tooth (CMT) Disease, Chronic Fatigue Syndrome (CFS), Chronic Functional Abdominal Pain (CFAP), Chronic Pain, Chronic Pancreatitis, Chronic Pelvic Pain Syndrome, Collapsed Lung (Pneumothorax), Complex Regional Pain Syndrome (RSD), Constipation, Corneal Neuropathic Pain, Crohn's Disease, Degenerative Disc Disease, Dental Pain, Dercum's Disease, Dermatomyositis, Diabetic Peripheral Neuropathy (DPN), Dry Eye Syndrome, Dystonia, Ehlers-Danlos Syndrome (EDS), Endometriosis, Eosinophilia-Myalgia Syndrome (EMS), Erythromelalgia, Fibromyalgia, Gout, Headaches, Herniated disc, Hydrocephalus, Inflammatory Bowel Disease (IBD), Intercostal Neuralgia, Interstitial Cystitis, Irritable Bowel syndrome (IBS), Juvenile Dermatositis (Dermatomyositis), Knee Injury, Leg Pain, Loin Pain-Haematuria Syndrome, Lupus, Lyme Disease, Medullary Sponge Kidney (MSK), Meralgia Paresthetica, Mesothelioma, Migraine, Musculoskeletal pain, Myofascial Pain, Myositis, Neck Pain, Neuropathic Pain, Occipital Neuralgia, ocular Itch, Osteoarthritis, Paget's Disease, Parsonage Turner Syndrome, Pelvic Pain, Periodontitis Pain, Peripheral Neuropathy, Phantom Limb Pain, Pinched Nerve, Polycystic Kidney Disease, Polymyalgia Rheumatica, Polymyositis, Porphyria, Post Herniorraphy Pain Syndrome, Post Mastectomy, Postoperative Pain, Pain Syndrome, Post Stroke Pain, Post Thorocotomy Pain Syndrome, Postherpetic Neuralgia (Shingles), Post-Polio Syndrome, Primary Lateral Sclerosis, Psoriatic Arthritis, Pudendal Neuralgia, Radiculopathy, Raynaud's Disease, Rheumatoid Arthritis (RA), Sacroiliac Joint Dysfunction, Sarcoidosi, Scheuemann's Kyphosis Disease, Sciatica, Scoliosis, Shingles (Herpes Zoster), Sjogren's Syndrome, Spasmodic Torticollis, Sphincter of Oddi Dysfunction, Spinal Cerebellum Ataxia (SCA Ataxia), Spinal Cord Injury, Spinal Stenosis, Syringomyelia, Tarlov Cysts, Transverse Myelitis, Trigeminal Neuralgia, Neuropathic Pain, Ulcerative Colitis, Vascular Pain or Vulvodynia.

In another embodiment, the itch associated condition is: chronic itch; contact dermatitis; Allergic blepharitis; Anemia; Atopic dermatitis; Bullous pemphigoid; Candidiasis; Chicken pox; end-stage renal failure; hemodialysis; Chronic urticaria; Contact dermatitis, Atopic Dermatitis; Dermatitis herpetiformis; Diabetes; Drug allergy, Dry Eye Syndrome; Dry skin; Dyshidrotic dermatitis; Ectopic eczema; Eosinophilic fasciitis; Epidermolysis bullosa; Erythrasma; Food allergy; Folliculitis; Fungal skin infection; Hemorrhoids; Herpes; HIV infection; Hodgkin's disease; Hyperthyroidism; lodinated contrast dye allergy; Iron deficiency anemia; Kidney disease; Leukemia, porphyrias; Lymphoma; Malignancy; Mastocystosis; Multiple myeloma; Neurodermatitis; Onchocerciasis; Paget's disease; Pediculosis; Polycythemia rubra vera; Prurigo nodularis; Lichen Planus; Lichen Sclerosis; Pruritus ani; Pseudorabies; Psoriasis; Rectal prolapse; Sarcoidosis granulomas; Scabies; Schistosomiasis; Scleroderma, Severe stress; Stasia dermatitis; Swimmer's itch; Thyroid disease; Tinea cruris; Rosacea; Cutaneous amyloidosis; Scleroderma; Acne; wound healing; burn healing; ocular itch; or Urticaria.

In a specific embodiment, the itch associated condition is urticaria, pruritus, atopic dermatitis, dry skin, psoriasis, contact dermatitis, or eczema.

In another embodiment, the ocular associated condition is: dry eye syndrome/keratoconjunctivitis sicca and related conditions, including xeropthalmia, meibomian gland dysfunction and lacrimal gland dysfunction; dry eye associated with other medical conditions including dacryoadenitis, dacryocystitis, allergic conjunctivitis, blepharitis, rheumatoid arthritis, systemic lupus erythematous, scleroderma, Sjogren's syndrome, Stevens-Johnson syndrome, sarcoidosis, sympathetic opthalmia, diabetic retinopathy, parasitic eye infections, thyroid disorders, and vitamin A deficiency; dry eye associated with medications such as antihistamines, decongestants, anti-depressants, tranquilizers, diuretics, hormone replacement, oral contraceptives, antihypertensives, isotretonin treatments for acne, and anticholinergic drugs; and dry eye associated with eye surgery including laser eye surgery, glaucoma surgery, corneal transplantation, and cataract removal surgery.

In another embodiment, cardiovascular and renal diseases associated condition is: peripheral vascular disease, cerebrovascular disease, coronary artery disease, cardiac hypertrophy, cardiac fibrosis, cardiovascular hypertension, renovascular hypertension, renal fibrosis, renal disease, nephritis, atherosclerosis, coronary atherosclerotic heart disease, acute myocardial infarction, stroke, thrombosis, coronary atherothrombosis, pulmonary embolism, myocardial ischemia, carotid stenosis, vertebral stenosis, intracranial stenosis, and aneurysms as well as treatment of cardiac dysfunction induced by sepsis, rheumatic fever, or other acute or chronic disorders that influence cardiovascular and renal function such as diabetes.

In another embodiment, the chronic inflammatory and autoimmune associated condition include: chronic pulmonary allergy, asthma, chronic bronchitis, atherosclerosis, Graves' disease, Hashimoto's thyroiditis, chronic inflammatory demyelinating polyneuropathy, ankylosing spondylitis, sacroiliitis, steatohepatitis, scleroderma, systemic sclerosis, diabetes, ulcerative colitis, Crohn's disease, inflammatory bowel disease, systemic lupus erythematous, alopecia areata, temporal arteritis, chronic peptic ulcer, polymyalgia rheumatica, periodontitis, sinusitis, rhinitis, pancreatitis, nephritis, Sjogren's syndrome, dermatomyositis, polymyositis, inclusion body myositis, autoimmune necrotizing myopathy, idiopathic inflammatory myopathies, multiple sclerosis, rheumatoid arthritis, and vasculitis.

As used herein, the term “autoimmune disorder”, or “inflammatory disorder” means a disease or disorder arising from and/or directed against an individual's own tissues or organs, or a co-segregate or manifestation thereof, or resulting condition therefrom. Typically, various clinical and laboratory markers of autoimmune diseases may exist including, but not limited to, hypergammaglobulinemia, high levels of autoantibodies, antigen-antibody complex deposits in tissues, clinical benefit from corticosteroid or immunosuppressive treatments, and lymphoid cell aggregates in affected tissues. Thus, in one embodiment, the method of present invention is provided to treat an autoimmune disorder, such as chronic inflammation, mast cell activation syndrome, Multiple Sclerosis, Steven Johnson's Syndrome, Toxic Epidermal Necrolysis, appendicitis, bursitis, cutaneous lupus, colitis, cystitis, dermatitis, phlebitis, reflex sympathetic dystrophy/complex regional pain syndrome (rsd/crps), rhinitis, tendonitis, tonsillitis, acne vulgaris, sinusitis, rosacea, psoriasis, graft-versus-host disease, reactive airway disorder, asthma, airway infection, allergic rhinitis, autoinflammatory disease, celiac disease, chronic prostatitis, diverticulitis, glomerulonephritis, hidradenitis suppurativa, hypersensitivities, intestinal disorder, epithelial intestinal disorder, inflammatory bowel disease, irritable bowel syndrome, Crohn's Disease, ulcerative colitis, lupus erythematous, interstitial cystitis, otitis, pelvic inflammatory disease, endometrial pain, reperfusion injury, rheumatic fever, rheumatoid arthritis, sarcoidosis, transplant rejection, psoriasis, lung inflammation, chronic obstructive pulmonary disease, permanent sputum eosiniophilia, eosinophilic leukemia, eosinophilic esophagitis, eosinophilic gastritis, mast cell gastrointestinal disease, hypereosinophilic syndrome, aspirin-exacerbated respiratory disease, nasal polyposis, chronic rhinosinusitis, antibody-dependent cell-mediated cytotoxicity, neurofibromatosis, swannamatoisis, tubulointerstitial nephritis, glomerulonephritis, diabetic nephropathy, allograft rejection, amyloidosis, renovascular ischemia, reflux nephropathy, polycystic kidney disease, liver fibrosis/cirrhosis, autoimmune liver disease, Biliary atresia, acute and chronic Hepatitis B and C virus, Liver tumors and cancer, Lung tumors and cancer, Alcoholic liver disease, Polycystic liver disease, Liver cholangiocarcinoma, neuromyelitis optica spectum disorder, cardiovascular disease, and vasculitis.

In another embodiment, cognitive impairment associated condition include neurodegenerative diseases including Parkinson's disease, Alzheimer's disease, Huntington's disease, Lewy body dementia, frontotemporal dementia, progressive supranuclear palsy, corticobasal syndrome, frontotemporal lobar degeneration, amyotrophic lateral sclerosis and multiple sclerosis, as well as age-induced cognitive impairment, vascular cognitive impairment and post-stroke cognitive impairment.

### EXAMPLES

All reagents, for which the synthesis is not described in the experimental part, are either commercially available, or are known compounds or may be formed from known compounds by known methods by a person skilled in the art. The compounds and intermediates produced according to the methods of the invention may require purification. Purification of organic compounds is well known to a person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using purpose-made or prepacked silica gel cartridges and eluents such as gradients of solvents such as heptane, ether, ethyl acetate, acetonitrile, ethanol and the like. In some cases, the compounds may be purified by preparative HPLC using methods as described.

Purification methods as described herein may provide compounds of the present invention which possess a sufficiently basic or acidic functionality in the form of a salt, such as, in the case of a compound of the present invention which is sufficiently basic, a trifluoroacetate or formate salt, or, in the case of a compound of the present invention, which is sufficiently acidic, an ammonium salt. A salt of this type can either be transformed into its free base or free acid form, respectively, by various methods known to a person skilled in the art or be used as salts in subsequent biological assays. It is to be understood that the specific form of a compound of the present invention as isolated and as described herein is not necessarily the only form in which said compound can be applied to a biological assay in order to quantify the specific biological activity.

Chemical names were generated using the naming function in ChemDraw software (Version 19.0.1.28) by PerkinElmer Informatics, Inc. In some cases, generally accepted names of commercially available reagents were used in place of names generated by the naming software.

### General Methods

¹H NMR (400 MHZ) spectra were obtained in solution of deuteriochloroform (CDCl₃), deuteriomethanol (CD₃OD) or dimethyl sulfoxide—D6 (DMSO).

HPLC retention times, purities, and mass spectra (LCMS) were obtained using one of the following methods.

Method 1: Shimadzu LC-20AB system equipped with Merck Chromolith Flash RP-18e, 5.0 mM, 3.0×25 mm column and LCMS-2020, using H2O with 0.0375% trifluoroacetic acid as the mobile phase A, and acetonitrile with 0.01875% trifluoroacetic acid as the mobile phase B. The gradient was 5-95% mobile phase B over 0.7 min then held at 95% mobile phase B for 0.4 min, then return to 5% mobile phase B for 0.5 min. The flow rate was 1.5 mL/min.

Method 2: Shimadzu LC-20AB system equipped with Welch Xtimate C18, 3.0 mM, 2.1×30 mm column and LCMS-2020, using H20 with 0.0375% trifluoroacetic acid as the mobile phase A, and acetonitrile with 0.01875% trifluoroacetic acid as the mobile phase B. The gradient was 10-80% mobile phase B over 3.0 min then held at 80% mobile phase B for 0.5 min, then return to 10% mobile phase B for 0.5 min. The flow rate was 1.2 mL/min.

All reactions were stirred magnetically, and temperatures are external reaction temperatures. Preparative HPLC purifications were typically performed eluting with various gradients of H₂O/ACN (0.1% formic acid).

The following abbreviations are used: ethyl acetate (EA), ethanol (EOH), dimethylformamide (DMF), petroleum ether (PE), triethylamine (TEA), 1-[Bis(dimethylamino)methylene]-1H-1,2,3-triazolo[4,5-b]pyridinium 3-oxid hexafluoro-phosphate (HATU), not determined (ND), and not applicable (NA), potassium terbutoxide (^{t}-BuOK).

### Example 1

### Synthesis of Compound 1-1

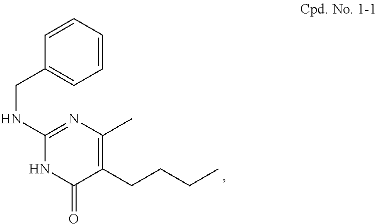 [SMILES: CCCCc1c(C)nc(NCc2ccccc2)[nH]c1=O]

and the like

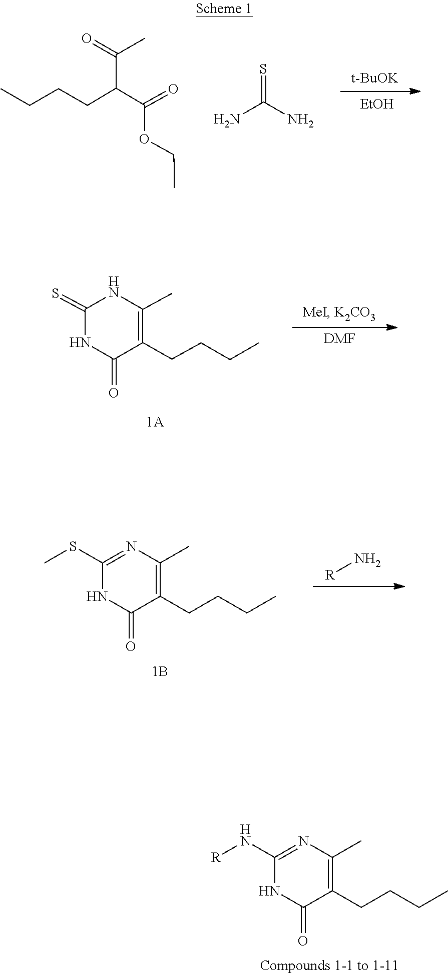

### Intermediate 1A

To the solution of ethyl 2-acetylhexanoate (10 g, 53.69 mmol, 1 eq) and thiourea (6.13 g, 80.54 mmol, 1.5 eq) in EtOH (100 mL) were added ^{t}-BuOK (12.05 g, 107.38 mmol, 2 eq). The mixture was then heated at 90° C. for 16 hours under N₂ atmosphere. LCMS showed the starting material was consumed and a new peak with the desired mass was detected. After return to room temperature, the reaction was quenched with water (100 mL) and HCl aqueous solution (40 mL, 1N). The precipitate was collected and dried. It was used as is in the next step. 5-butyl-6-methyl-2-thioxo-1H-pyrimidin-4-one (9.93 g, 50.05 mmol, 93.23% yield) was obtained as a white solid. 1H NMR (400 MHZ, DMSO-d₆) δ=2.257-2.098; (m, 2H), 2.098; (s, 3H), 1.32-1.254; (m, 4H), 0.864; (t, J=6.8 Hz, 3H). LCMS-ESI (m/z) calculated for C₉H₁₄N₂OS: 198.08; found 199.3 [M+H]⁺, t_{R}=0.775 min (Method 1).

### Intermediate 1B

To a solution of 5-butyl-6-methyl-2-thioxo-1H-pyrimidin-4-one (5 g, 25.22 mmol, 1 eq) and K₂CO₃ (6.97 g, 50.43 mmol, 2 eq), Mel (2.86 g, 20.17 mmol, 1.26 mL, 0.8 eq) in DMF (80 mL) was added. The reaction was stirred at 25° C. for 2 h under N₂ atmosphere. LCMS showed the starting material was consumed and a new peak with the desired mass was detected. The reaction mixture was added to H₂O (100mL). The precipitate was collected and dried in vacuo to give the title compound. It was used in the next step without purification. 5-butyl-4-methyl-2-methylsulfanyl-1H-pyrimidin-6-one (4.67 g, 22.00 mmol, 87.23% yield) was obtained as a white solid. LCMS-ESI (m/z) calculated for C₁₀H₁₆N₂OS: 212.10; found 213.2 [M+H]⁺, t_{R}=0.828 min (Method 1).

### Compound 1-1

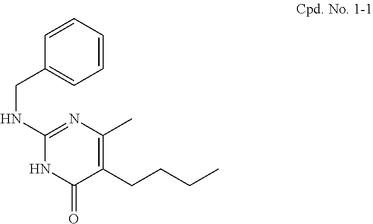 [SMILES: CCCCc1c(C)nc(NCc2ccccc2)[nH]c1=O]

To the solution of 5-butyl-4-methyl-2-methylsulfanyl-1H-pyrimidin-6-one (100 mg, 471.01 μmol, 1 eq) in o-xylene (1 mL) was added phenylmethanamine (252.35 mg, 2.36 mmol, 256.71 μL, 5 eq). The reaction was capped and stirred at 140° C. for 12 h under N₂ atmosphere. After return to room temperature, the reaction mixture was concentrated, and the product purified by reverse-phase HPLC. 2-(benzylamino)-5-butyl-4-methyl-1H-pyrimidin-6-one was obtained as a white solid (32.5 mg, 117.49 μmol, 24.95% yield, 98.1% purity). ¹H NMR (400 MHZ, DMSO-d₆) δ=7.36-7.22; (m, 5H), 6.62; (br s, 1H), 4.44; (d, J=5.8 Hz, 2H), 2.26; (br t, J=7.2 Hz, 2H), 2.06; (s, 3H), 1.36-1.26; (m, 4H), 0.92-0.86; (m, 3H) MS (ESI): m/z 272.2 [M+1]+. LCMS-ESI (m/z) calculated for C₁₆H₂₁N₃O: 271.17; found 272.2 [M+H]⁺, t_{R}=1.926 min (Method 2).

The compounds listed in Table 1 were made using the procedures of Scheme 1 and starting from the appropriate amine.

TABLE 1

| | | | | HPLC | | |
|---|---|---|---|---|---|---|
| | | | | Retention | | |
| Cpd. | | Yield | | Time | observed | Purity |
| No. | R-NH₂ | (%) | ¹H NMR | (minutes) | [M + H]⁺ | Method |
| 1-2 | 3-methoxy | 33.25 | ¹H NMR (400 MHz, CD₃OD) δ = 14.04-13.42 | 1.388 | 302.2 | 2 |
| | phenyl | | (m, 1H), 9.58 (br s, 1H), 7.26-7.20 (m, 1H), | | | |
| | methanamine | | 6.96 (br s, 2H), 6.84 (br d, J = 8.0 Hz, 1H), 4.76 | | | |
| | | | (br s, 2H), 3.78 (s, 3H), 2.46-2.10 (m, 5H), | | | |
| | | | 1.46-1.14 (m, 4H), 0.92-0.74 (m, 3H) | | | |
| 1-3 | 2-methoxy | 46.07 | ¹H NMR (400 MHz, CD₃OD) δ = 7.33-7.24 (m, | 1.877 | 302.2 | 2 |
| | phenyl | | 2H), 7.00 (d, J = 8.1 Hz, 1H), 6.92 (t, J = 7.4 Hz, | | | |
| | methanamine | | 1H), 4.52 (s, 2H), 4.58-4.47 (m, 1H), 3.94- | | | |
| | | | 3.84 (m, 3H), 2.40 (t, J = 7.4 Hz, 2H), 2.20 (s, | | | |
| | | | 3H), 1.52-1.26 (m, 4H), 1.04-0.88 (m, 3H). | | | |
| 1-4 | m-tolyl | 9.01 | ¹H NMR (400 MHz, DMSO-d₆) δ = 10.54 (br s, | 2.262 | 286.2 | 2 |
| | methanamine | | 1H), 7.26-7.18 (m, 1H), 7.16-7.04 (m, 3H), | | | |
| | | | 6.54 (br s, 1H), 4.40 (d, J = 5.8 Hz, 2H), 2.28 (s, | | | |
| | | | 3H), 2.28-2.20 (m, 2H), 2.06 (s, 3H), 1.36- | | | |
| | | | 1.22 (m, 4H), 0.92-0.84 (m, 3H) | | | |
| 1-5 | o-tolyl | 29.3 | ¹H NMR (400 MHz, CD₃OD) δ = 7.34-7.28 (m, | 1.439 | 286.2 | 2 |
| | methanamine | | 1H), 7.25 (t, J = 3.4 Hz, 2H), 7.24-7.20 (m, | | | |
| | | | 1H), 4.65 (s, 2H), 2.58-2.44 (m, 2H), 2.38 (d, | | | |
| | | | J = 1.6 Hz, 6H), 1.57-1.32 (m, 4H), 1.04-0.92 | | | |
| | | | (m, 2H), 1.04-0.92 (m, 1H). | | | |
| 1-6 | 1-phenyl | 25.48 | ¹H NMR (400 MHz, DMSO-d₆) = 10.23 (br s, | 1.963 | 286.1 | 2 |
| | ethanamine | | 1H), 7.38-7.32 (m, 4H), 7.27-7.22 (m, 1H), | | | |
| | | | 6.64 (br s, 1H), 5.04 (quin, J = 7.2 Hz, 1H), 2.24 | | | |
| | | | (br t, J = 7.1 Hz, 2H), 2.03 (s, 3H), 1.41 (d, J = 6.9 | | | |
| | | | Hz, 3H), 1.31-1.23 (m, 4H), 0.89-0.84 (m, | | | |
| | | | 3H). | | | |
| 1-7 | 4- | 20.24 | ¹H NMR (400 MHz, DMSO-d₆) δ = 10.45 (br s, | 1.615 | 315.2 | 2 |
| | (aminomethyl) - | | 1H), 7.15 (d, J = 8.6 Hz, 2H), 6.69 (d, J = 8.8 Hz, | | | |
| | N,N-dimethyl- | | 2H), 6.38 (br s, 1H), 4.28 (d, J = 5.5 Hz, 2H), | | | |
| | aniline | | 2.86 (s, 6H), 2.26 (br t, J = 7.0 Hz, 2H), 2.07 (s, | | | |
| | | | 3H), 1.33-1.24 (m, 4H), 0.90-0.85 (m, 3H). | | | |
| 1-8 | 3- | 37.01 | ¹H NMR (400 MHz, DMSO-d₆) δ = 10.52 (br s, | 1.801 | 315.2 | 2 |
| | (aminomethyl)- | | 1H), 7.14 (t, J = 7.8 Hz, 1H), 6.70 (s, 1H), 6.64- | | | |
| | N,N-dimethyl- | | 6.58 (m, 1H), 6.52 (br s, 1H), 4.36 (d, J = 5.8 | | | |
| | aniline | | Hz, 2H), 2.88 (s, 6H), 2.26 (br t, J = 7.2 Hz, 2H), | | | |
| | | | 2.06 (s, 3H), 1.36-1.24 (m, 4H), 0.92-0.84 | | | |
| | | | (m, 3H). | | | |
| 1-9 | 2- | 26.23 | ¹H NMR (400 MHz, CD₃OD) δ = 7.35 (d, J = 7.5 | 2.041 | 315.2 | 2 |
| | (aminomethyl)- | | Hz, 1H), 7.33-7.23 (m, 2H), 7.15-7.06 (m, | | | |
| | N,N-dimethyl- | | 1H), 4.55 (s, 2H), 2.83-2.70 (m, 6H), 2.47- | | | |
| | aniline | | 2.35 (m, 2H), 2.19 (s, 3H), 1.51-1.29 (m, 4H), | | | |
| | | | 1.02-0.88 (m, 3H). | | | |
| 1-10 | 4-methoxy | 28.77 | ¹H NMR (400 MHz, CD₃OD) δ = 7.35-7.19 (m, | 1.371 | 302.1 | 2 |
| | phenyl) | | 2H), 6.97-6.81 (m, 2H), 4.46 (s, 2H), 3.84- | | | |
| | methanamine | | 3.75 (m, 3H), 2.47-2.34 (m, 2H), 2.26 (s, 1H), | | | |
| | | | 2.21 (s, 2H), 1.53-1.33 (m, 4H), 1.03-0.88 | | | |
| | | | (m, 3H) | | | |
| 1-11 | aniline | 3.49 | ¹H NMR (400 MHz, CD₃OD) δ = 8.15 (br s, 1H), | 0.959 | 258.1 | 2 |
| | | | 7.61-7.61 (m, 1H), 7.58 (dd, J = 1.0, 8.6 Hz, | | | |
| | | | 1H), 7.37-7.28 (m, 2H), 7.12-7.05 (m, 1H), | | | |
| | | | 2.49-2.41 (m, 2H), 2.30-2.23 (m, 3H), 1.51- | | | |
| | | | 1.37 (m, 4H), 1.00-0.94 (m, 3H) | | | |

### Example 2

### Synthesis of Compound 2-1

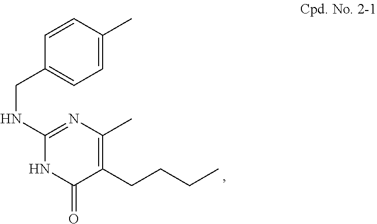 [SMILES: CCCCc1c(C)nc(NCc2ccc(C)cc2)[nH]c1=O]

and the like

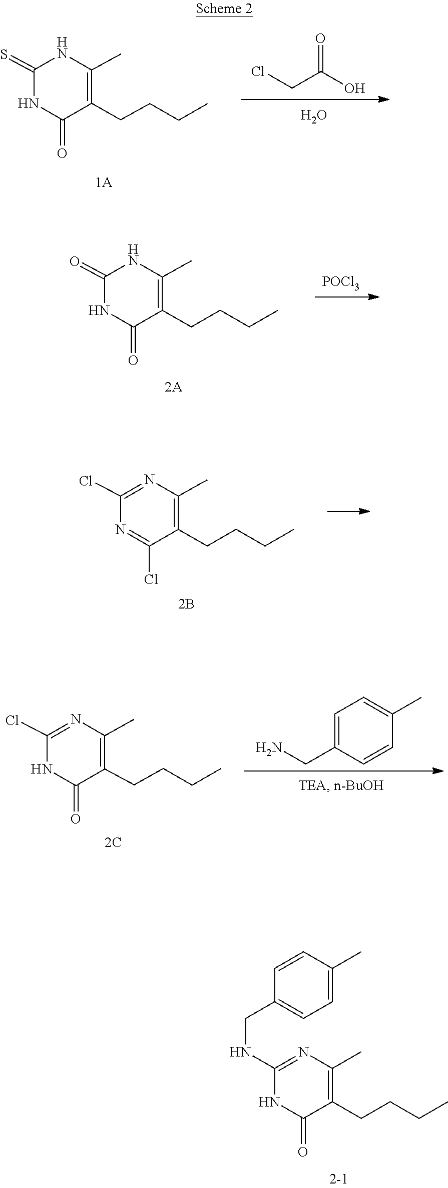

### Intermediate 2A: 5-butyl-6-methyl-1H-pyrimidine-2,4-dione

To a solution of 5-butyl-6-methyl-2-thioxo-1H-pyrimidin-4-one (20.5 g, 103.39 mmol, 1 eq) in H₂O (200 ml) was added 2-chloroacetic acid (24.42 g, 258.47 mmol, 29.08 mL, 2.5 eq) at 25° C. The reaction was stirred at 100° C. for 12 h under N₂ atmosphere. After return to room temperature, the reaction mixture was concentrated in vacuo. The residue (18.5 g, 101.53 mmol, 98.20% yield) was used for next step directly without purification. LCMS-ESI (m/z) calculated for C₉H₁₄N₂O₂: 182.11; found 183.3 [M+H]⁺, t_{R}=0.727 min (Method 1).

### Intermediate 2B: 5-butyl-2,4-dichloro-6-methyl-pyrimidine

A solution of 5-butyl-6-methyl-1H-pyrimidine-2,4-dione (1.32 g, 7.24 mmol, 1 eq) in POCl₃ (22.21 g, 144.88 mmol, 13.46 mL, 20 eq) was heated at 110° C. for 4 h under N₂ atmosphere. After return to room temperature, the crude was poured in water. The formed precipitate was filtered and dried to obtain the desired compound. The crude product (1.45 g, 6.62 mmol, 91.35% yield) was used for next step directly without purification. LCMS-ESI (m/z) calculated for C₉H₁₂Cl₂N₂: 218.04; found 219.0 [M+H]⁺, t_{R}=1.457 min (Method 2).

### Intermediate 2C: 5-butyl-2-chloro-4-methyl-1H-pyrimidin-6-one

To a solution of 5-butyl-2,4-dichloro-6-methyl-pyrimidine (1.45 g, 6.62 mmol, 1 eq) in H₂O (25 mL) was added NaOH (5 M, 15.88 mL, 12 eq). The mixture was refluxed for 1 hour at 100° C. under N₂ atmosphere. After return to room temperature, the solution was acidified with 1M HCl (aq). until pH=3-4, then extracted with EA (30 mL×2). The organic layers were combined and washed with brine (50 mL), dried over Na₂SO₄ and concentrated under reduced pressure. The residue was purified by flash silica gel chromatography. (SiO₂, 10% EA in PE) to afford 5-butyl-2-chloro-4-methyl-1H-pyrimidin-6-one (300 mg, 1.50 mmol, 22.59% yield) as a white solid. LCMS-ESI (m/z) calculated for C₉H₁₃ClN₂O: 200.07; found 201.1 [M+H]⁺, t_{R}=0.783 min (Method 1).

### Compound 2-1

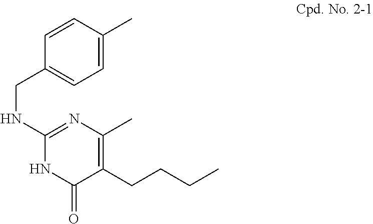 [SMILES: CCCCc1c(C)nc(NCc2ccc(C)cc2)[nH]c1=O]

To a solution of 5-butyl-2-chloro-4-methyl-1H-pyrimidin-6-one (80 mg, 398.67 μmol, 1 eq) in n-BuOH (2 mL) was added TEA (201.71 mg, 1.99 mmol, 277.45 μL, 5 eq) and p-tolylmethanamine (96.62 mg, 797.35 μmol, 100.75 μL, 2 eq). The mixture was refluxed for 12 hours at 130° C. After return to room temperature, the mixture was concentrated in vacuo to give a residue. The product was purified by prep-HPLC. Compound 5-butyl-4-methyl-2-(p-tolylmethylamino)-1H-pyrimidin-6-one was obtained as a white solid (24.3 mg, 83.81 μmol, 21.02% yield, 98.43% purity). ¹H NMR (400 MHZ, CD₃OD) δ=7.24-7.18; (m, 2H), 7.11; (d, J=7.9 Hz, 2H), 4.66; (s, 2H), 2.46-2.38; (m, 2H), 2.31; (s, 3H), 2.19; (s, 3H), 1.48-1.33; (m, 4H), 0.99-0.90; (m, 3H). LCMS-ESI (m/z) calculated for C₁₇H₂₃N₃O: 285.391; found 286.2 [M+H]⁺, t_{R}=1.360 min (Method 2).

The compounds listed in Table 2 were made using the procedures of Scheme 2.

TABLE 2

| | | | | HPLC | | |
|---|---|---|---|---|---|---|
| | | | | Retention | | |
| Cpd. | | Yield | | Time | observed | Purity |
| No. | R-NH₂ | (%) | ¹H NMR | (minutes) | [M + H]⁺ | Method |
| 2-2 | 4- | 51.73 | ¹H NMR (400 MHz, DMSO-d₆) δ = 10.82 (s, | 1.195 | 297.1 | 2 |
| | (aminomethyl) | | 1H), 8.62 (s, 1H), 8.26-8.24 (m, 1H), 8.27- | | | |
| | benzonitrile | | 8.24 (m, 1H), 8.24 (d, J = 8.3 Hz, 1H), 8.03 (br | | | |
| | | | t, J = 5.9 Hz, 1H), 7.87 (d, J = 8.3 Hz, 2H), 5.14- | | | |
| | | | 5.06 (m, 1H), 5.05 (d, J = 5.9 Hz, 2H), 3.80 (br | | | |
| | | | s, 8H), 2.82-2.67 (m, 2H), 2.54 (s, 1H), 2.50 | | | |
| | | | (s, 3H), 1.84-1.73 (m, 1H), 1.40-1.30 (m, | | | |
| | | | 3H). | | | |
| 2-3 | 3- | 32.09 | ¹H NMR (400 MHz, DMSO-d₆) δ = 10.35 (s, | 1.168 | 297.2 | 2 |
| | (aminomethyl) | | 1H), 7.69 (d, J = 7.5 Hz, 1H), 7.65 (s, 1H), 7.62- | | | |
| | benzonitrile | | 7.49 (m, 3H), 4.56 (d, J = 5.9 Hz, 2H), 2.30 (br | | | |
| | | | s, 2H), 2.04 (s, 3H), 1.33 (br d, J = 3.3 Hz, 4H), | | | |
| | | | 0.97-0.81 (m, 3H). | | | |

### Example 3

### Synthesis of Compound 3-1

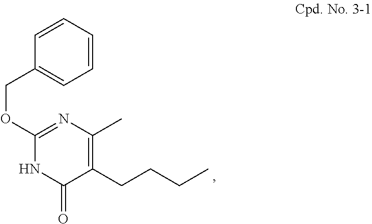 [SMILES: CCCCc1c(C)nc(OCc2ccccc2)[nH]c1=O]

and the like

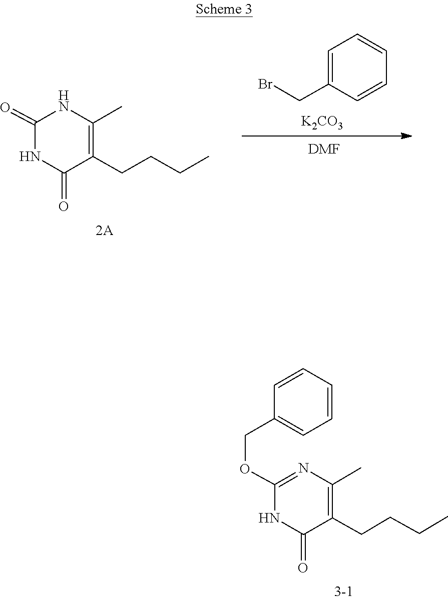

### Compound 3-1: 2-benzyloxy-5-butyl-4-methyl-1H-pyrimidin-6-one

To the solution of bromomethylbenzene (469.31 mg, 2.74 mmol, 325.91 μL, 1 eq) in DMF (5 mL) and K2CO3 (758.46 mg, 5.49 mmol, 2 eq) was added 5-butyl-6-methyl-1H-pyrimidine-2,4-dione (500 mg, 2.74 mmol, 1 eq). The mixture was stirred at 70° C. for 12 h under N₂ atmosphere. After return to room temperature, the reaction mixture was acidified with HCl in Dioxane to pH=3-4. The precipitate was filtered and purified by reversed-phase HPLC. Compound 2-benzyloxy-5-butyl-4-methyl-1H-pyrimidin-6-one (26.1 mg, 91.33 μmol, 3.33% yield, 95.3% purity) was obtained as a greenish solid. ¹H NMR (400 MHZ, DMSO-d6) δ=11.36; (s, 1H), 7.38-7.32; (m, 2H), 7.28-7.24; (m, 1H), 7.16; (d, J=7.4 Hz, 2H), 5.08; (s, 2H), 2.28; (br t, J=7.2 Hz, 2H), 2.10; (s, 3H), 1.32-1.24; (m, 4H), 0.88-0.82; (m, 3H). LCMS-ESI (m/z) calculated for C₁₆H₂₀N₂O₂: 272.15; found 273.2 [M+H]⁺, t_{R}=1.857 min (Method 2).

### Example 4

### Synthesis of Compound 4-1

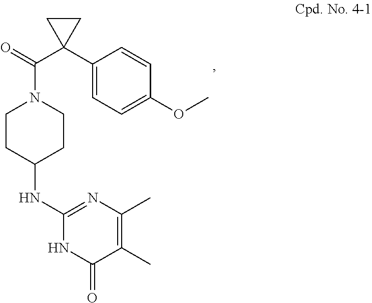 [SMILES: COc1ccc(C2(C(=O)N3CCC(Nc4nc(C)c(C)c(=O)[nH]4)CC3)CC2)cc1]

and the like

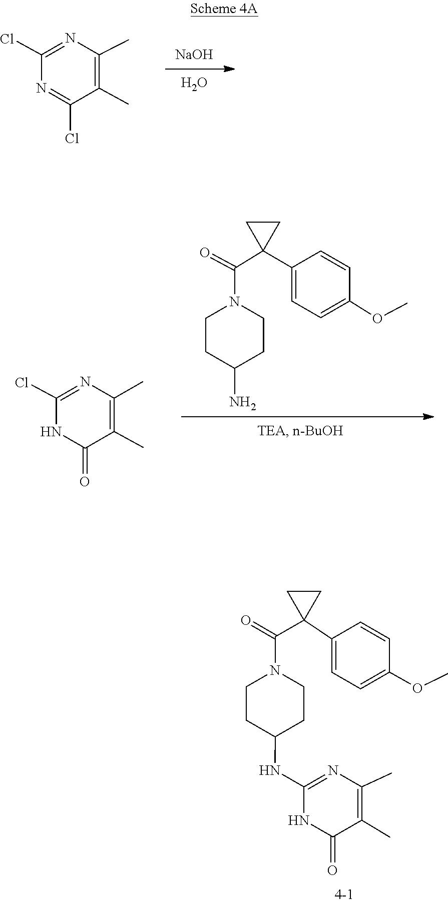

### Intermediate 4A: 2-chloro-4,5-dimethyl-1H-pyrimidin-6-one

To a solution of 2,4-dichloro-5,6-dimethyl-pyrimidine (1 g, 5.65 mmol, 1 eq) in H₂O (20 mL) was added NaOH (5 M, 13.76 mL, 12.18 eq). The reaction was then stirred at 100° C. for 1 h under N₂ atmosphere. LCMS showed the starting material was consumed and a new peak with the desired mass was detected. The reaction mixture was acidified with 1M HCl to pH=3-4, then extracted with EA (25 mL×2). The organic layers were combined, washed with brine (50 mL), dried over Na₂SO₄ and concentrated under reduced pressure. 2-chloro-4,5-dimethyl-1H-pyrimidin-6-one (590 mg, 3.72 mmol, 65.86% yield) was obtained as a white solid and used as is in the next step. LCMS-ESI (m/z) calculated for C₆H₇ClN₂O: 158.59; found 159.0 [M+H]⁺, t_{R}=0.324 min (Method 2).

### Compound 4-1: 2-[[1-[1-(4-methoxyphenyl)cyclo-propanecarbonyl]-4-piperidyl]amino]-4,5-dimethyl-1H-pyrimidin-6-one

To a solution of (4-amino-1-piperidyl)-[1-(4-methoxyphenyl)cyclopropyl]-methanone (100 mg, 364.49 μmol, 1 eq) in n-BuOH (2 mL) were added TEA (184.41 mg, 1.82 mmol, 253.66 μL, 5 eq) and 2-chloro-4,5-dimethyl-1H-pyrimidin-6-one (115.60 mg, 728.98 μmol, 2 eq). The mixture was stirred for 24 hours at 130° C. under N₂ atmosphere. After return to rt, the mixture was concentrated in vacuo to give a residue that was purified by reverse prep-HPLC. The titled compound 2-[[1-[1-(4-methoxyphenyl)cyclopropane-carbonyl]-4-piperidyl]amino]-4,5-dimethyl-1H-pyrimidin-6-one (30.3 mg, 75.02 μmol, 20.58% yield, 98.17% purity) was obtained as a white solid. ¹H NMR (400 MHZ, CD₃OD) δ=7.16; (d, J=8.6 Hz, 2H), 6.89; (d, J=8.8 Hz, 2H), 4.38; (br d, J=10.1 Hz, 1H), 4.15; (br d, J=11.1 Hz, 1H), 4.01-3.86; (m, 1H), 3.77; (s, 3H), 3.18-2.84; (m, 2H), 2.15; (s, 3H), 2.08-1.93; (m, 1H), 1.88; (s, 3H), 1.75; (br d, J=10.0 Hz, 1H), 1.45-1.17; (m, 4H), 1.13-0.87; (m, 1H), 1.13-0.84; (m, 1H). LCMS-ESI (m/z) calculated for C₂₂H₂₈N₄O₃: 396.491; found 397.2 [M+H]⁺, t_{R}=1.270 min (Method 2).

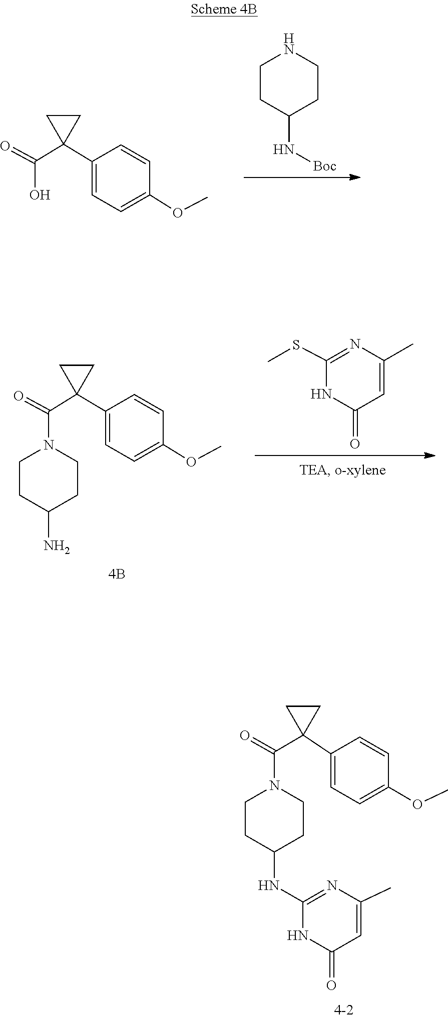

### Intermediate 4B: (4-amino-1-piperidyl)-[1-(4-methoxy-phenyl)cyclopropyl]methanone

A solution of 1-(4-methoxyphenyl)cyclopropanecarboxylic acid (500 mg, 2.60 mmol, 1 eq) in DCM (5 mL) was added to SOCl₂ (3.09 g, 26.01 mmol, 1.89 mL, 10 eq) and DMF (19.01 mg, 260.13 μmol, 20.01 μL, 0.1 eq). The reaction mixture was then refluxed for 3 hours with stirring at 40° C. under N₂ atmosphere. The reaction mixture was concentrated under reduced pressure and the residue used for next step directly without purification.

To a solution of tert-butyl N-(4-piperidyl) carbamate (475.36 mg, 2.37 mmol, 1 eq) and TEA (480.35 mg, 4.75 mmol, 660.73 μL, 2 eq) in DCM (5 mL) was added 1-(4-methoxyphenyl)cyclopropanecarbonyl chloride (0.5 g, 2.37 mmol, 1 eq) in DCM (5 mL). The reaction was stirred at 25°° C. for 12 h under N2 atmosphere. The reaction mixture was concentrated under reduced pressure to remove solvent. The residue was diluted with water (20 mL) and extracted with EA (20 mL×3). The combined organic layers were washed with brine (30 mL), dried over Na₂SO₄ and concentrated in vacuo. The residue was purified by flash silica gel chromatography. (SiO2, eluent: EA:PE 3:7) to give tert-butyl N-[1-[1-(4-methoxyphenyl) cyclopropanecarbonyl]-4-piperidyl]carbamate (807 mg, 2.16 mmol, 90.79% yield) as a white solid. LCMS-ESI (m/z) calculated for C₂₁H₃₀N₂O₄: 374.22 found 375.2 [M+H]⁺, t_{R}=1.487 min (Method 2).

A solution of tert-butyl N-[1-[1-(4-methoxyphenyl)cyclopropanecarbonyl]-4-piperidyl]carbamate (807 mg, 2.16 mmol, 1 eq) in HCl/dioxane (4 M, 4.04 mL, 7.5 eq) was stirred at 25° C. for 1 hour under N₂ atmosphere. The reaction was then stirred with EA at 25° C. for 30 min, then the product was collected by filtration, washed with EA and dried in vacuo to give (4-amino-1-piperidyl)-[1-(4-methoxyphenyl)-cyclopropyl]methanone (520 mg, 1.67 mmol, 77.63% yield, HCl) as a white solid. LCMS-ESI (m/z) calculated for C₁₆H₂₂N₂O₂: 274.17; found 275.2 [M+H]⁺, t_{R}=0.664 min (Method 1).

### Compound 4-2: 2-[[1-[1-(4-methoxyphenyl) cyclo-propanecarbonyl]-4-piperidyl]amino]-4-methyl-1H-pyrimidin-6-one

To the solution of (4-amino-1-piperidyl)-[1-(4-methoxyphenyl)cyclopropyl]methanone (660 mg, 2.41 mmol, 5.01 eq) in o-xylene (1 mL) were added 4-methyl-2-methylsulfanyl-1H-pyrimidin-6-one (75 mg, 480.14 μmol, 1 eq) and TEA (242.92 mg, 2.40 mmol, 334.14 μL, 5 eq). The reaction mixture was stirred at 140° C. for 12 h. After return to room temperature, the reaction mixture was acidified by HCl in Dioxane to pH=3-4. The precipitate was filtered, and the product purified by reverse-phase HPLC. 2-[[1-[1-(4-methoxyphenyl) cyclopropanecarbonyl]-4-piperidyl]amino]-4-methyl-1H-pyrimidin-6-one (66.8 mg, 171.17 μmol, 35.65% yield, 98.0% purity) was obtained as a yellow solid. ¹H NMR (400 MHZ, CD₃OD) δ=7.24-7.12; (m, 2H), 6.88; (d, J=7.8 Hz, 2H), 6.02; (br s, 1H), 4.56-4.14; (m, 2H), 4.02; (br s, 1H), 3.78; (s, 3H), 3.16-2.80 (m, 2H), 2.34; (s, 3H), 2.08-1.72; (m, 2H), 1.54-1.06; (m, 6H) MS (ESI): m/z 383.2 [M+1]+. LCMS-ESI (m/z) calculated for C₂₁H₂₆N₄O₃: 382.464; found 383.2 [M+H]⁺, t_{R}=1.111 min (Method 2).

The compounds listed in Table 4 were made using the procedures of Scheme 4A or 4B, starting from the appropriate dichloropyrimidine.

TABLE 4

| | | | | Retention | | |
|---|---|---|---|---|---|---|
| Cpd. | | Yield | | Time | observed | Purity |
| No. | Product Name | (%) | ¹H NMR | (minutes) | [M + H]⁺ | Method |
| 4-2 | 2-((1-(1-(4- | 12.88 | ¹H NMR (400 MHz, CD₃OD) δ = 8.16 (s, 1H), | 1.015 | 383.2 | 2 |
| | methoxyphenyl)cyclo- | | 7.32-7.04 (m, 2H), 6.97-6.71 (m, 2H), | | | |
| | propane-1- | | 5.55 (s, 1H), 4.44-4.36 (m, 1H), 4.33- | | | |
| | carbonyl)piperidin-4- | | 4.31 (m, 1H), 4.53-4.31 (m, 1H), 4.20 (br | | | |
| | yl)amino)-6- | | d, J = 12.1 Hz, 1H), 4.14-4.03 (m, 1H), 3.92- | | | |
| | methylpyrimidin- | | 3.89 (m, 1H), 3.79 (s, 1H), 3.12-2.74 (m, | | | |
| | 4(3H)-one | | 2H), 2.11 (s, 3H), 2.00-1.64 (m, 2H), 1.49- | | | |
| | | | 1.18 (m, 3H), 1.21-0.75 (m, 1H), 1.18- | | | |
| | | | 0.72 (m, 1H), 1.20-0.67 (m, 1H) | | | |
| 4-3 | 2-[[1-[1-(4- | 25.02 | ¹H NMR (400 MHz, CD₃OD) δ = 7.60 (d, | 1.158 | 369.2 | 2 |
| | methoxyphenyl)cyclo- | | J = 6.8 Hz, 1H), 7.34-7.08 (m, 2H), 7.00- | | | |
| | propanecarbonyl]-4- | | 6.76 (m, 2H), 5.73 (d, J = 6.8 Hz, 1H), 5.94- | | | |
| | piperidyl]amino]-1H- | | 5.46 (m, 1H), 4.48-4.07 (m, 2H), 4.55- | | | |
| | pyrimidin-6-one | | 4.04 (m, 1H), 4.01-3.85 (m, 1H), 3.79 (s, | | | |
| | | | 3H), 3.22-2.77 (m, 2H), 2.05-1.66 (m, | | | |
| | | | 2H), 1.47-1.21 (m, 4H), 1.18-0.92 (m, | | | |
| | | | 2H) | | | |
| 4-4 | 2-[1-[1-(4- | 23.45 | Not acquired | 0.729 | 383.2 | 1 |
| | methoxyphenyl)cyclo- | | | | | |
| | propanecarbonyl]-4- | | | | | |
| | piperidyl]amino]-5- | | | | | |
| | methyl-1H-pyrimidin- | | | | | |
| | 6-one | | | | | |
| 4-5 | 5-fluoro-2-[[1-[1-(4- | 14.40 | ¹H NMR (400 MHz, CD₃OD) δ = 7.58 (br s, | 1.276 | 387.1 | 2 |
| | methoxyphenyl)cyclo- | | 1H), 7.16 (br d, J = 8.4 Hz, 2H), 6.88 (br d, | | | |
| | propanecarbonyl]-4- | | J = 8.5 Hz, 2H), 4.39 (br s, 1H), 4.15 (br s, | | | |
| | piperidyl]amino]-1H- | | 1H), 3.99-3.83 (m, 1H), 3.77 (s, 3H), 3.18- | | | |
| | pyrimidin-6-one | | 2.76 (m, 2H), 2.15-1.61 (m, 2H), 1.57- | | | |
| | | | 1.22 (m, 4H), 1.16-0.73 (m, 2H) | | | |

### Example 5

### Synthesis of Compound 5-1

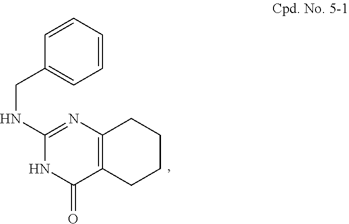 [SMILES: O=c1[nH]c(NCc2ccccc2)nc2c1CCCC2]

and the like

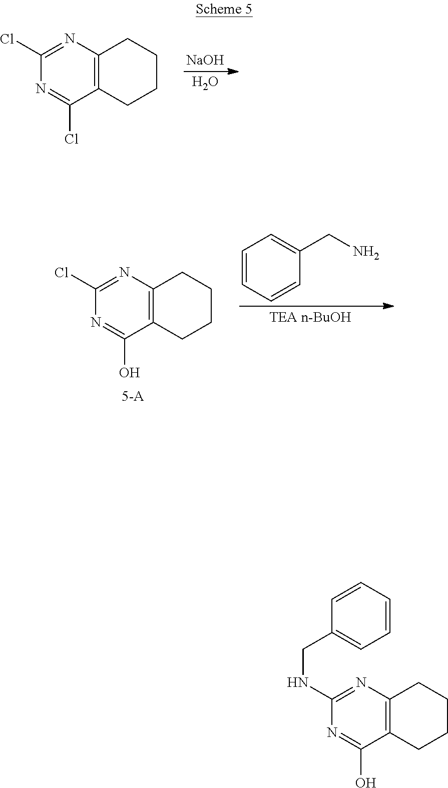

### Intermediate 5A: 2-chloro-5,6,7,8-tetrahydro-3H-quinazolin-4-one

To a solution of 2,4-dichloro-5,6,7,8-tetrahydroquinazoline (1 g, 4.92 mmol, 1 eq) in H₂O (18 mL) was added NaOH (5 M, 12.00 mL, 12.18 eq). The reaction mixture was stirred at 100° C. for 1 h under N2 atmosphere. Once all the starting material was consumed, the reaction was cooled down to room temperature. The crude was acidified with an aqueous solution of 1M HCl to pH 3-4, then extracted with EA (50ml×2). The organic layers were combined and washed with brine (100 mL), dried over Na₂SO4 and concentrated. The obtained white solid, 2-chloro-5,6,7,8-tetrahydro-3H-quinazolin-4-one (500 mg, 2.49 mmol, 50.60% yield, 92% purity), was used in the next step without further purification. LCMS-ESI (m/z) calculated for C₈H₉ClN₂O: 184.04; found 185.2 [M+H]⁺, t_{R}=0.681 min (Method 1).

### Compound 5-1: 2-(benzylamino)-5,6,7,8-tetrahydro-3H-quinazolin-4-one

To a solution of phenylmethanamine (232.15 mg, 2.17 mmol, 236.17 μL, 2 eq) in n-BuOH (3 mL) was added TEA (582.07 mg, 5.75 mmol, 800.64 μL, 5.31 eq) and 2-chloro-5,6,7,8-tetrahydro-3H-quinazolin-4-one (200 mg, 1.08 mmol, 1 eq). The mixture was heated at 130° C. for 12 h under N₂ atmosphere. Once all the starting material had disappeared, the reaction mixture was acidified with an aqueous solution of 1M HCl to pH 3-4. The precipitate was filtered, and the filtrate purified by reversed-phase HPLC. 2-(benzylamino)-5,6,7,8-tetrahydro-3H-quinazolin-4-one (63.2 mg, 235.16 μmol, 21.71% yield, 95% purity) was obtained as a white solid. ¹H NMR (400 MHZ, CD₃OD) δ=7.38-7.32; (m, 4H), 7.30-7.24; (m, 1H), 4.55; (s, 3H), 2.49; (t, J=6.0 Hz, 2H), 2.36; (t, J=5.8 Hz, 2H), 1.82-1.70; (m, 4H); MS (ESI): m/z 256.1 [M+1]+. LCMS-ESI (m/z) calculated for C₁₅H₁₇N₃O: 255.14; found 256.1 [M+H]⁺, t_{R}=1.008 min (Method 2).

### Example 6

### Synthesis of Compound 6-1

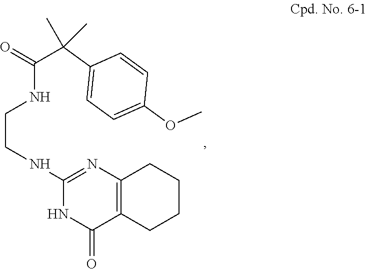 [SMILES: COc1ccc(C(C)(C)C(=O)NCCNc2nc3c(c(=O)[nH]2)CCCC3)cc1]

and the like

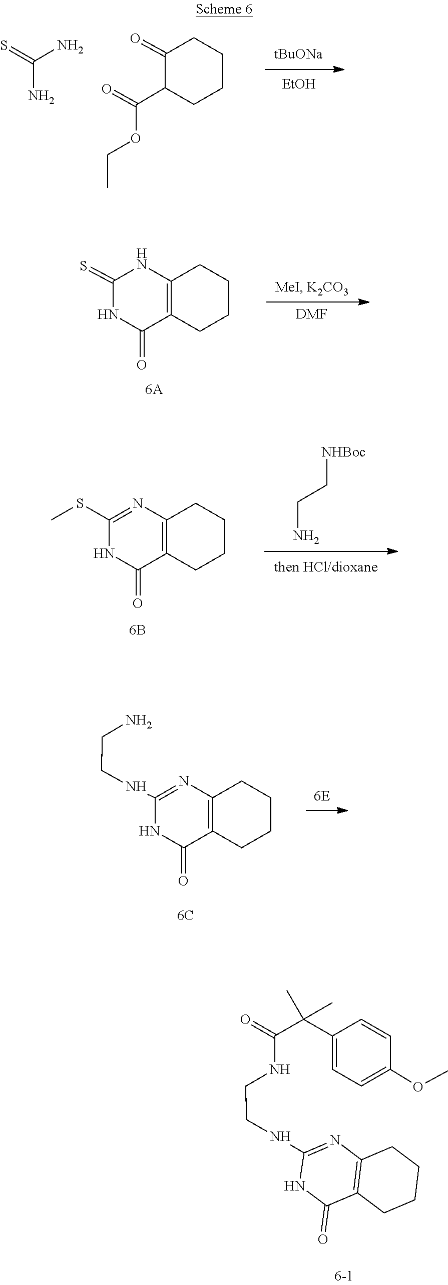

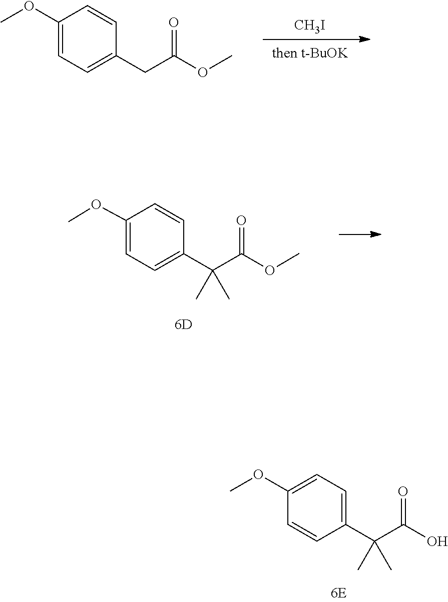

### Intermediate 6A: 2-thioxo-5,6,7,8-tetrahydro-1H-quinazolin-4-one

To a solution of thiourea (8.94 g, 117.51 mmol, 1 eq) and ethyl 2-oxocyclohexanecarboxylate (20 g, 117.51 mmol, 18.87 mL, 1 eq) in EtOH (100 mL) was added ^{I}-BuONa (22.59 g, 235.01 mmol, 2 eq). The mixture was stirred at 90° C. for 12 h. After return to room temperature, the reaction mixture was filtered. The product, a yellow solid, was used without further purification (20 g, 109.74 mmol, 93.39% yield) was obtained as yellow solid. LCMS-ESI (m/z) calculated for C₂₂H₃₀N₄O₃: 182.24; found 183.2 [M+H]⁺, t_{R}=0.566 min (Method 1).

### Intermediate 6B: 2-methylsulfanyl-5,6,7,8-tetrahydro-3H-quinazolin-4-one

To a solution of 2-thioxo-5,6,7,8-tetrahydro-1H-quinazolin-4-one (19 g, 104.26 mmol, 1 eq) and K₂CO₃ (28.82 g, 208.51 mmol, 2 eq) in DMF (180 mL) was added Mel (11.84 g, 83.41 mmol, 5.19 mL, 0.8 eq). The reaction was stirred at 25° C. for 2 h under N₂ atmosphere then quenched with H₂O (30ml) and concentrated under reduced pressure. The precipitate was washed with EA (20 ml) and H₂O (20 ml) and used as is in the next step. The product 2-methylsulfanyl-5,6,7,8-tetrahydro-3H-quinazolin-4-one (16 g, 81.52 mmol, 78.19% yield) was obtained as white solid. LCMS-ESI (m/z) calculated for C₉H₁₂N₂OS: 196.07; found 197.3 [M+H]+, t_{R}=0.729 min (Method 1).

### Intermediate 6C: 2-(2-aminoethylamino)-5,6,7,8-tetrahydro-3H-quinazolin-4-one

A solution of tert-butyl N-[2-[(4-oxo-5,6,7,8-tetrahydro-3H-quinazolin-2-yl)amino]ethyl] carbamate (120 mg, 389.14 μmol, 1 eq) in HCl/dioxane (2 mL) was stirred at 25° C. for 0.5 h. The reaction mixture was then concentrated under reduced pressure to give the expected product 2-(2-aminoethylamino)-5,6,7,8-tetrahydro-3H-quinazolin-4-one as a yellow solid. It was used as is in the next step.

### Intermediate 6D: methyl 2-(4-methoxyphenyl)-2-methyl-propanoate

To a solution of methyl 2-(4-methoxyphenyl)acetate (1 g, 5.55 mmol, 877.19 μL, 1 eq) in THF (10 mL) was added dropwise CH₃I (2.36 g, 16.65 mmol, 1.04 mL, 3 eq) at −78° C. Then ^{t}-BuOK (1 M, 16.65 mL, 3 eq) was added. The resulting mixture was stirred at −78° C. for 1 hr, followed by 25° C. for another 1 h. TLC showed a new spot was formed. The reaction mixture was quenched with H₂O (10 mL) and extracted with EA (15 mL×2). The combined organic layers were washed with brine (30 ml×2), dried over Na₂SO4, filtered and concentrated under reduced pressure. The crude product was used in the next step without further purification. Compound methyl 2-(4-methoxyphenyl)-2-methyl-propanoate (900 mg, 4.32 mmol, 77.88% yield) was obtained as yellow oil. ¹H NMR (400 MHz, CDCl₃) δ=7.11; (d, J=10 Hz, 2H), 6.71; (d, J=10 Hz, 2H), 3.64; (br s, 3H), 3.50; (br s, 3H), 1.41; (s, 6H)

### Intermediate 6E: 2-(4-methoxyphenyl)-2-methyl-propanoic acid

To a solution of methyl 2-(4-methoxyphenyl)-2-methyl-propanoate (500 mg, 2.40 mmol, 1 eq) and lithium hydroxide hydrate (100.75 mg, 2.40 mmol, 1 eq) in THF (5 mL) were added MeOH (2 mL) and H₂O (1 mL). The resulting mixture was stirred at 25° C. for 16 hours. TLC (PE:EA 5:1) showed the starting material was consumed completely. The reaction mixture was concentrated, and the crude product used in the next step without further purification.

### Compound 6-1: 2-(4-methoxyphenyl)-2-methyl-N-[2-1(4-oxo-5,6,7,8-tetrahydro-3H-quinazolin-2-yl)aminolethyl]propanamide

To a solution of 2-(4-methoxyphenyl)-2-methyl-propanoic acid (93.26 mg, 480.17 μmol, 1 eq), HATU (365.15 mg, 960.34 μmol, 2 eq) and DIEA (124.12 mg, 960.34 μmol, 167.27 μl, 2 eq) in DMF (3 mL) was added 2-(2-aminoethylamino)-5,6,7,8-tetrahydro-3H-quinazolin-4-one (100 mg, 480.17 μmol, 1 eq). The mixture was stirred at 25° C. for 12 h. The reaction mixture was concentrated under reduced pressure to give a residue that was purified by prep-HPLC. The product, 2-(4-methoxyphenyl)-2-methyl-N-[2-[(4-oxo-5,6,7,8-tetrahydro-3H-quinazolin-2-yl)amino]ethyl]propanamide (14.5 mg, 37.71 μmol, 7.85% yield) was obtained as a white solid. ¹H NMR (400 MHZ, CD₃OD) δ=6.80-6.78; (m, 1H), 6.78-6.71; (m, 2H), 6.39-6.30; (m, 2H), 3.03-2.96; (m, 2H), 2.92; (d, J=6.1 Hz, 2H), 2.89-2.87; (m, 3H), 2.24; (s, 1H), 2.01-1.94; (m, 2H), 1.89; (br t, J=5.3 Hz, 2H), 1.31; (br s, 4H), 1.04; (s, 6H). LCMS-ESI (m/z) calculated for C₂₁H₂₈N₄O₃: 384.480; found 385.6 [M+H]⁺, t_{R}=0.634 min (Method 2).

### Example 7

### Synthesis of Compound 7-1

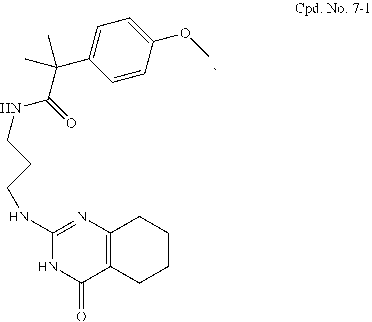 [SMILES: COc1ccc(C(C)(C)C(=O)NCCCNc2nc3c(c(=O)[nH]2)CCCC3)cc1]

and the like

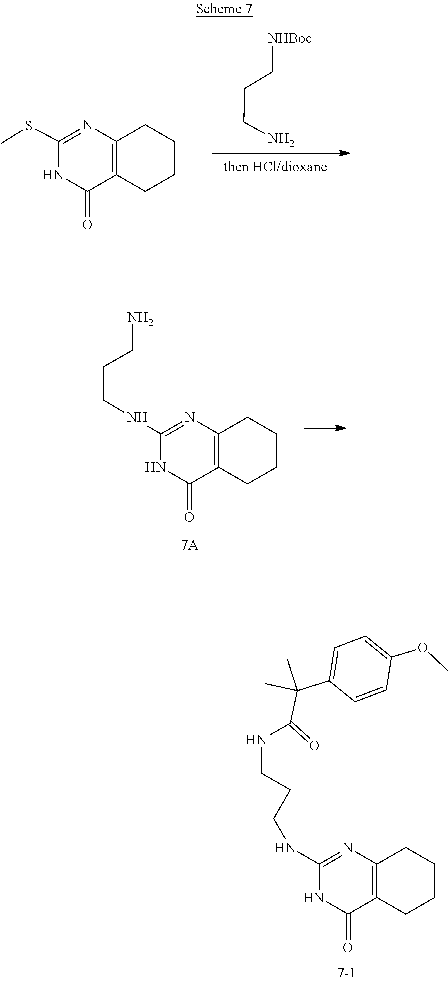 [SMILES: CSc1nc2c(c(=O)[nH]1)CCCC2 ; CC(C)(C)OC(=O)NCCCN ; NCCCNc1nc2c(c(=O)[nH]1)CCCC2 ; COc1ccc(C(C)(C)C(=O)NCCCNc2nc3c(c(=O)[nH]2)CCCC3)cc1]

### Intermediate: tert-butyl N-[3-[(4-oxo-5,6,7,8-tetrahydro-3H-quinazolin-2-yl)-amino]propyl]carbamate

2-methylsulfanyl-5,6,7,8-tetrahydro-3H-quinazolin-4-one (100 mg, 509.50 μmol, 1 eq), tert-butyl N-(3-aminopropyl)carbamate (443.88 mg, 2.55 mmol, 444.77 μL, 5 eq) were heated in a microwave neat at 140° C. for 3 hours. The reaction mixture was then quenched with H₂O (10 mL) and extracted with EA (15 mL×2). The combined organic layers were washed with brine (30 ml×2), dried over Na₂SO₄, filtered and concentrated under reduced pressure. The crude product (150 mg) was used in the next step without further purification. LCMS-ESI (m/z) calculated for C₁₆H₂₆N₄O₃: 322.40; found 323.2 [M+H]⁺, t_{R}=1.017 min (Method 2).

### Intermediate 7A: 2-(3-aminopropylamino)-5,6,7,8-tetrahydro-3H-quinazolin-4-one

A solution of tert-butyl N-[3-[(4-oxo-5,6,7,8-tetrahydro-3H-quinazolin-2-yl)amino]propyl]carbamate (150 mg, 465.26 μmol, 1 eq) in citric acid (3 mL) was stirred at 25° C. for 1 hour. The reaction progress was followed by TLC (PE:EA 3:1). The reaction mixture was concentrated and used in the next step without further purification.

### Compound 7-1: 2-(4-methoxyphenyl)-2-methyl-N-[3-[(4-oxo-5,6,7,8-tetrahydro-3H-quinazolin-2-yl)amino]propyl]propanamide

A solution of 2-(4-methoxyphenyl)-2-methyl-propanoic acid (131.07 mg, 674.81 μmol, 1.5 eq), DIEA (116.28 mg, 899.74 μmol, 156.72 μL, 2 eq) and HATU (256.58 mg, 674.81 μmol, 1.5 eq) in DMF (1 mL) was stirred at 25° C. for 0.5 hours. 2-(3-aminopropylamino)-5,6,7,8-tetrahydro-3H-quinazolin-4-one (100 mg, 449.87 μmol, 1 eq) was added and the resulting mixture was stirred at 25° C. for 16 hours. The reaction mixture was concentrated, and the crude product was purified by reversed prep-HPLC. 2-(4-methoxyphenyl)-2-methyl-N-[3-[(4-oxo-5,6,7,8-tetrahydro-3H-quinazolin-2-yl)amino]-propyl]propanamide (36 mg, 90.34 μmol, 20.08% yield, 100% purity) was obtained as white solid. ¹H NMR (400 MHZ, CD₃OD) δ=7.38-7.26; (m, 3H), 6.93-6.86; (m, 2H), 3.79; (s, 3H), 3.30-3.20; (m, 4H), 2.43-2.37; (m, 2H), 2.34; (br t, J=5.6 Hz, 2H), 1.77-1.64; (m, 6H), 1.53; (s, 6H). LCMS-ESI (m/z) calculated for C₂₂H₃₀N₄O₃: 398.507; found 399.3 [M+H]⁺, t_{R}=1.298 min (Method 2).

### Example 8

### Synthesis of Compound 8-1

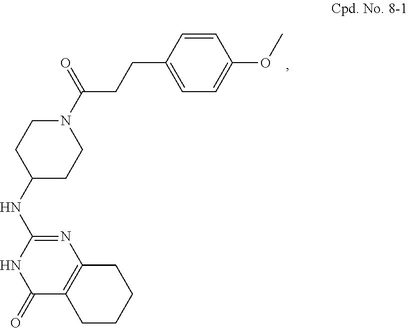 [SMILES: COc1ccc(CCC(=O)N2CCC(Nc3nc4c(c(=O)[nH]3)CCCC4)CC2)cc1]

and the like

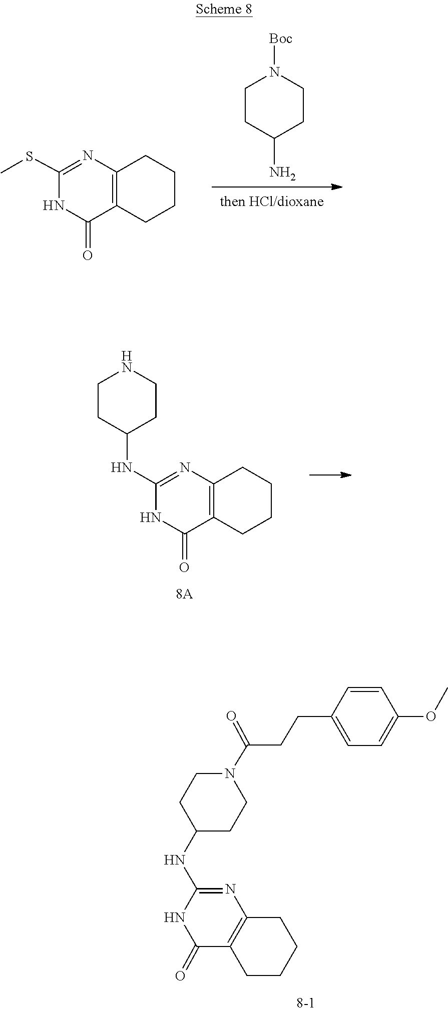 [SMILES: CSc1nc2c(c(=O)[nH]1)CCCC2 ; CC(C)(C)OC(=O)N1CCC(N)CC1 ; O=c1[nH]c(NC2CCNCC2)nc2c1CCCC2 ; COc1ccc(CCC(=O)N2CCC(Nc3nc4c(c(=O)[nH]3)CCCC4)CC2)cc1]

### Intermediate 8A: 2-(4-piperidylamino)-5,6,7,8-tetrahydro-3H-quinazolin-4-one

A mixture of 2-methylsulfanyl-5,6,7,8-tetrahydro-3H-quinazolin-4-one (1.50 g, 7.64 mmol, 1 eq) and tert-butyl 4-aminopiperidine-1-carboxylate (7.65 g, 38.21 mmol, 5 eq) was stirred at 140° C. for 6 hr in a microwave reactor. The residue was diluted with EA (100 mL), washed with an aqueous citric acid solution (150 mL×3) followed by a brine solution (100 mL). The organic layer was dried over anhydrous Na₂SO₄ and concentrated under reduced pressure. The crude product was used for next step directly without purification. LCMS-ESI (m/z) calculated for C₁₈H₂₈NO₃: 348.22; found 349.2 [M+H]⁺, t_{R}=1.220 min (Method 2).

To a solution of tert-butyl 4-[(4-oxo-5,6,7,8-tetrahydro-3H-quinazolin-2-yl)amino]piperidine-1-carboxylate (1.65 g, 4.74 mmol, 1 eq) in dioxane (10 mL) was added HCl in dioxane (4 M, 15 mL, 12.67 eq). Then the resulting mixture was stirred at 25° C. for 1 hr. The reaction mixture was concentrated in vacuum and the product purified by reverse prep-HPLC. 2-(4-piperidylamino)-5,6,7,8-tetrahydro-3H-quinazolin-4-one (710 mg, 2.86 mmol, 60.38% yield) was obtained as a yellow solid. LCMS-ESI (m/z) calculated for C₁₃H₂₀N₄O: 248.16; found 249.2 [M+H]⁺, t_{R}=0.553 min (Method 2).

### Compound 8-1: 2-[[1-[3-(4-methoxyphenyl)-propanoyl]-4-piperidyl]amino]-5,6,7,8-tetrahydro-3H-quinazolin-4-one

To a solution of 3-(4-methoxyphenyl)propanoic acid (72.57 mg, 402.70 μmol, 1 eq) in DMF (2 mL) were added HATU (229.68 mg, 604.05 μmol, 1.5 eq) and DIEA (104.09 mg, 805.40 μmol, 140.29 uL, 2 eq). The reaction mixture was stirred at 25° C. for 30 min. 2-(4-piperidylamino)-5,6,7,8-tetrahydro-3H-quinazolin-4-one (150 mg, 604.05 μmol, 1.5 eq) was added and the resulting reaction mixture was stirred at 25° C. for 12 h. The reaction was quenched by adding water (50 mL) to the mixture. The product was extracted with EA (50 mL×3). The combined organic layers were washed with water (30 mL), brine (30 mL), dried over Na₂SO₄, filtered and concentrated under reduced pressure to give a residue. The product was purified by reverse-phase HPLC. 2-[[1-[3-(4-methoxyphenyl)propanoyl]-4-piperidyl]amino]-5,6,7,8-tetrahydro-3H-quinazolin-4-one was isolated as a white solid (53.7 mg, 130.81 μmol, 32.48% yield). ¹H NMR (400 MHZ, CD3OD) δ=8.08; (s, 1H), 7.18-7.10; (m, J=8.4 Hz, 2H), 6.84; (d, J=8.6 Hz, 2H), 4.44; (br d, J=13.0 Hz, 1H), 4.00-3.84; (m, 2H), 3.76; (s, 3H), 3.16-3.04; (m, 1H), 2.92-2.76; (m, 3H), 2.74-2.62; (m, 2H), 2.52; (br t, J=5.6 Hz, 2H), 2.36 (br t, J=5.8 Hz, 2H), 2.00-1.92; (m, 2H), 1.84-1.68; (m, 4H), 1.44-1.24; (m, 2H) LCMS-ESI (m/z) calculated for C₂₃H₃₀N₄O₃: 410.231; found 411.3 [M+H]⁺, t_{R}=1.239 min (Method 2).

The compounds listed in Table 8 were made using the procedures of Scheme 8 and starting from the appropriate acid.

TABLE 8

| | | | | HPLC | | |
|---|---|---|---|---|---|---|
| | | | | Retention | | |
| Cpd. | | Yield | | Time | observed | Purity |
| No. | Product | (%) | ¹H NMR | (minutes) | [M + H]⁺ | Method |
| 8-2 | 4-[1,1-dimethyl-2-oxo- | 5.68 | ¹H NMR (400 MHz, CD₃OD) δ 7.76 (d, | 1.232 | 420.3 | 2 |
| | 2-[4-[(4-oxo-5,6,7,8- | | J = 8.4 Hz, 2H), 7.46 (d, J = 8.4 Hz, 2H), | | | |
| | tetrahydro-3H- | | 4.67-4.32 (m, 2H), 4.01-3.77 (m, 1H), | | | |
| | quinazolin-2- | | 2.94-2.70 (m, 2H), 2.55-2.43 (m, 2H), | | | |
| | yl)amino]-1- | | 2.33 (br t, J = 5.8 Hz, 2H), 2.23-1.65 (m, | | | |
| | piperidyl]ethyl]benzonitrile | | 6H), 1.55 (s, 6H), 1.40-0.71 (m, 2H) | | | |
| 8-3 | 2-[[1-(4- | 19.43 | ¹H NMR (400 MHz, DMSO-d₆) δ 10.43 (br | 1.880 | 383.2 | 2 |
| | methoxybenzoyl)-4- | | s, 1H), 7.35 (d, J = 8.8 Hz, 2H), 6.98 (d, | | | |
| | piperidyl]amino]- | | J = 8.6 Hz, 2H), 6.21 (br s, 1H), 4.17 (br s, | | | |
| | 5,6,7,8-tetrahydro-3H- | | 1H), 4.05-3.86 (m, 1H), 3.79 (s, 3H), | | | |
| | quinazolin-4-one | | 3.21-2.97 (m, 2H), 2.40-2.28 (m, 2H), | | | |
| | | | 2.19 (br t, J = 5.8 Hz, 2H), 1.87 (br s, 2H), | | | |
| | | | 1.73-1.51 (m, 4H), 1.37 (br d, J = 9.6 Hz, | | | |
| | | | 2H) | | | |
| 8-4 | 2-[1-(2-methyl-2- | 20.50 | ¹H NMR (400 MHz, DMSO-d₆) δ 10.21 (br | 1.863 | 395.2 | 2 |
| | phenyl-propanoyl)-4- | | s, 1H), 7.43-7.30 (m, 2H), 7.26-7.14 (m, | | | |
| | piperidyl]amino]- | | 3H), 6.09 (br s, 1H), 4.24 (br s, 1H), 3.81- | | | |
| | 5,6,7,8-tetrahydro-3H- | | 3.60 (m, 1H), 2.74 (br s, 2H), 2.28 (br t, | | | |
| | quinazolin-4-one | | J = 5.6 Hz, 2H), 2.16 (br t, J = 5.6 Hz, 2H), | | | |
| | | | 2.01-1.66 (m, 1H), 1.66-1.50 (m, 5H), | | | |
| | | | 1.42 (s, 7H), 1.29-0.52 (m, 1H) | | | |
| 8-5 | 2-[[1-[2-(4- | 15.87 | ¹H NMR (400 MHz, DMSO-d₆) δ 7.15 (d, | 1.946 | 397.2 | 2 |
| | methoxyphenyl)acetyl | | J = 8.6 Hz, 2H), 6.87 (d, J = 8.6 Hz, 2H), 4.18 | | | |
| | ]-4-piperidyl]amino]- | | (br d, J = 12.8 Hz, 1H), 3.85 (br d, J = 13.6 | | | |
| | 5,6,7,8-tetrahydro-3H- | | Hz, 2H), 3.72 (s, 3H), 3.63 (d, J = 2.6 Hz, | | | |
| | quinazolin-4-one | | 2H), 3.12 (br t, J = 11.4 Hz, 1H), 2.79 (br t, | | | |
| | | | J = 11.4 Hz, 1H), 2.39-2.28 (m, 2H), 2.19 | | | |
| | | | (br t, J = 5.8 Hz, 2H), 1.91-1.73 (m, 2H), | | | |
| | | | 1.61 (td, J = 6.8, 13.5 Hz, 4H), 1.30-1.08 | | | |
| | | | (m, 2H) | | | |
| 8-6 | 2-[[1-[2-(4- | 26.18 | ¹H NMR (400 MHz, DMSO-d₆) δ 10.29 (br | 2.337 | 429.2 | 2 |
| | chlorophenyl)-2- | | s, 1H), 7.42 (d, J = 8.8 Hz, 2H), 7.23 (d, | | | |
| | methyl-propanoyl]-4- | | J = 8.8 Hz, 2H), 6.19 (br s, 1H), 4.26 (br s, | | | |
| | piperidyl]amino]- | | 1H), 3.68-3.84 (m, 1H), 2.86-2.67 (m, | | | |
| | 5,6,7,8-tetrahydro-3H- | | 2H), 2.29 (br t, J = 5.4 Hz, 2H), 2.17 (br t, | | | |
| | quinazolin-4-one | | J = 5.6 Hz, 2H), 2.03-1.70 (m, 1H), 1.68- | | | |
| | | | 1.50 (m, 5H), 1.41 (s, 7H), 1.28-0.54 (m, | | | |
| | | | 1H) | | | |
| 8-7 | 2-[[1-[1-(4- | 23.96 | ¹H NMR (400 MHz, DMSO-d₆) δ 7.14- | 2.083 | 423.2 | 2 |
| | methoxyphenyl)cyclop | | 7.04 (m, 2H), 6.92-6.82 (m, 2H), 4.18 (br | | | |
| | ropanecarbonyl]-4- | | s, 1H), 3.94-3.75 (m, 2H), 3.72 (s, 3H), | | | |
| | piperidyl]amino]- | | 3.05-2.75 (m, 2H), 2.33 (br s, 2H), 2.18 | | | |
| | 5,6,7,8-tetrahydro-3H- | | (br t, J = 5.6 Hz, 2H), 1.95-1.49 (m, 6H), | | | |
| | quinazolin-4-one | | 1.36-0.84 (m, 6H) | | | |

### Example 9

### Synthesis of Compound 9-1

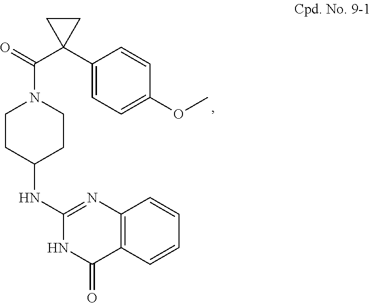 [SMILES: COc1ccc(C2(C(=O)N3CCC(Nc4nc5ccccc5c(=O)[nH]4)CC3)CC2)cc1]

and the like

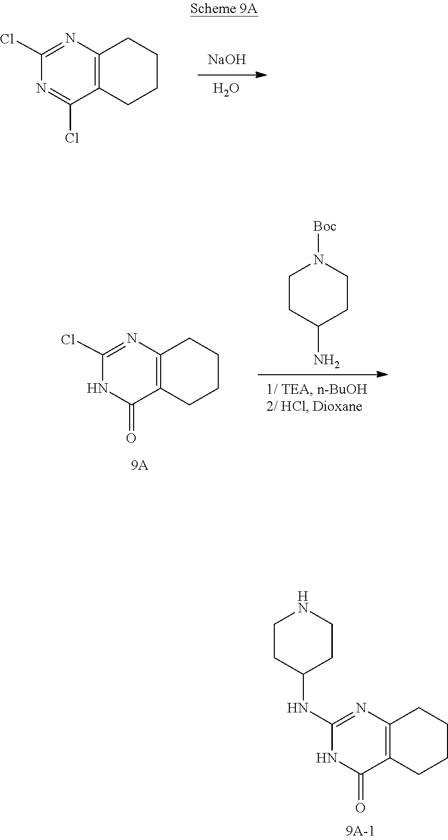

### Intermediate 9A: 2-chloro-5,6,7,8-tetrahydro-3H-quinazolin-4-one

To a solution of 2,4-dichloro-5,6,7,8-tetrahydroquinazoline (2 g, 9.85 mmol, 1 eq) in H₂O (36 mL) was added NaOH (5 M, 24.00 mL, 12.18 eq), then the reaction was stirred 100° C. for 1 h under N₂ atmosphere. After return to room temperature, the reaction mixture was acidified to pH 3-4 with aqueous 1M HCl. The product was extracted with EA (50 mL×2). The combined organic layers were washed with brine (100 mL), dried over Na₂SO₄ and concentrated under reduced pressure. The crude product was used for next step directly without purification. 2-chloro-5,6,7,8-tetrahydro-3H-quinazolin-4-one (1.52 g, crude) was obtained as a brown solid. LCMS-ESI (m/z) calculated for C₈H₉ClN₂O: 184.04; found 185.2 [M+H]⁺, t_{R}=0.690 min (Method 1).

### Intermediate 9A-1: 2-(4-piperidylamino)-5,6,7,8-tetrahydro-3H-quinazolin-4-one

To a solution of 2-chloro-5,6,7,8-tetrahydro-3H-quinazolin-4-one (1.42 g, 7.69 mmol, 1 eq) in n-BuOH (20 mL) was added tert-butyl 4-aminopiperidine-1-carboxylate (2.31 g, 11.54 mmol, 1.5 eq) and TEA (3.89 g, 38.46 mmol, 5.35 mL, 5 eq). The reaction was stirred at 120° C. for 14 h under N₂ atmosphere. Then tert-butyl 4-aminopiperidine-1-carboxylate (2.31 g, 11.54 mmol, 1.5 eq) and TEA (3.89 g, 38.46 mmol, 5.35 mL, 5 eq) was added and the reaction was stirred at 130° C. for 14 more hours under N₂ atmosphere. The reaction was concentrated in vacuo, the mixture was triturated with EA, then filtered. The filter cake was dissolved in MeOH and concentrated in vacuo. Tert-butyl 4-[(4-oxo-5,6,7,8-tetrahydro-3H-quinazolin-2-yl)amino]piperidine-1-carboxylate (2.83 g, crude) was obtained as a white solid. LCMS-ESI (m/z) calculated for C₁₈H₂₈N₄O₃: 348.22; found 349.4 [M+H]⁺, t_{R}=0.760 min (Method 1).

Deprotection was performed as previously described.

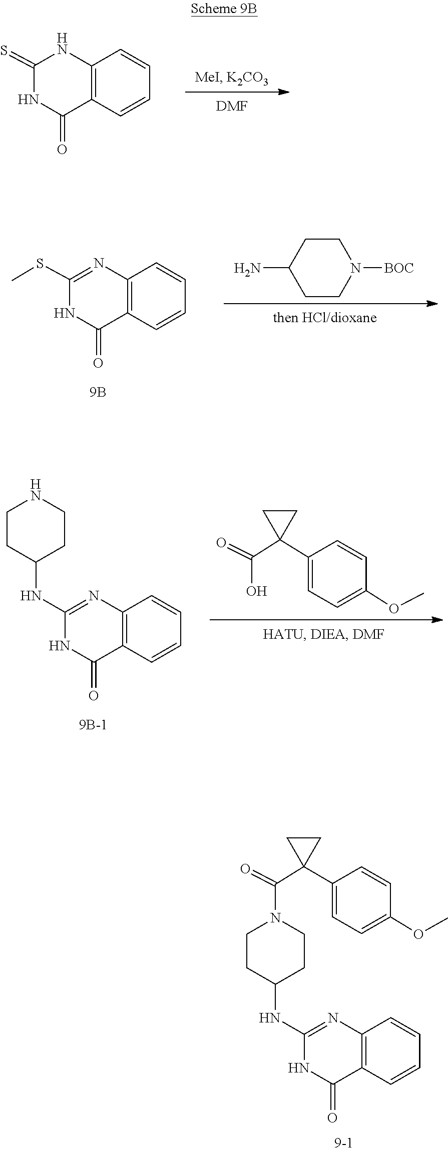

### Intermediate 9B: 2-methylsulfanyl-3H-quinazolin-4-one

To a solution of 2-thioxo-1H-quinazolin-4-one (4.5 g, 25.25 mmol, 1 eq) and K₂CO₃ (6.98 g, 50.50 mmol, 2 eq) in DMF (50 mL) was added CH₃I (2.87 g, 20.20 mmol, 1.26 mL, 0.8 eq). The mixture was stirred at 25° C. for 3 h. The reaction mixture pH was adjusted to 5 with a NaHCO3 solution and extracted with EA. The combined organic layers were washed with brine (50 mL), dried over anhydrous Na2SO4, and concentrated under reduced pressure. The crude product was used for next step directly without purification. Compound 2-methylsulfanyl-3H-quinazolin-4-one (4.6 g, crude) was obtained as a yellow solid. ¹H NMR (400 MHZ, CDCl₃) δ 8.26; (d, J=7.2 Hz, 1H), 7.73; (t, J=7.2 Hz, 1H), 7.62; (d, J=7.2 Hz, 1H), 7.41; (t, J=7.2 Hz, 1H), 3.50; (s, 3H). LCMS-ESI (m/z) calculated for C₉H₈N₂OS: 192.04; found 193.2 [M+H]⁺, t_{R}=0.763 min (Method 1).

### Intermediate 9B-1: 2-(4-piperidylamino)-3H-quinazolin-4-one

A mixture of 2-methylsulfanyl-3H-quinazolin-4-one (1 g, 5.20 mmol, 1 eq) and tert-butyl 4-aminopiperidine-1-carboxylate (8.33 g, 41.62 mmol, 8 eq) was heated at 140° C. for 12 h in a microwave reactor. The residue was then diluted with EA (100 mL) and extracted with citric acid (150 ml×3). The organic layer was washed with brine, dried over anhydrous Na₂SO₄, and concentrated under reduced pressure. The crude product was used for next step directly without purification. Compound tert-butyl 4-[(4-oxo-3H-quinazolin-2-yl)amino]piperidine-1-carboxylate (2.12 g, crude) was obtained as a white solid. ¹H NMR (400 MHZ, DMSO-d₆) δ 8.168; (br s, 1H), 7.87; (d, J=7.6 Hz, 1H), 7.55; (t, J=7.2 Hz, 1H), 7.24; (d, J=8.0 Hz, 1H), 7.09; (t, J=7.6 Hz, 1H), 6.84; (br s, 1H), 4.03-3.7;5 (m, 4H), 3.12-2.95; (m, 1H), 2.74; (d, J=15 Hz, 2H), 2.64; (d, J=15 Hz, 2H), 1.40; (s, 9H). LCMS-ESI (m/z) calculated for C₁₈H₂₄N₄O₃: 344.18; found 345.2 [M+H]+, t_{R}=1.268 min (Method 2).

A solution of tert-butyl 4-[(4-oxo-3H-quinazolin-2-yl)amino]piperidine-1-carboxylate (150 mg, 435.53 μmol, 1 eq) in HCl/dioxane (4 M, 3.00 mL, 27.55 eq) was stirred at 25° C. for 4 h. The reaction mixture was concentrated under reduced pressure to give a residue. The crude product was used for next step directly without purification. Compound 2-(4-piperidylamino)-3H-quinazolin-4-one (100 mg, crude, HCl) was obtained as a white solid.

### Compound 9-1: 2-[[1-[1-(4-methoxyphenyl)-cyclopropane carbonyl]-4-piperidyl]amino]-3H-quinazolin-4-one

To a solution of 1-(4-methoxyphenyl)cyclopropanecarboxylic acid (205.39 mg, 1.07 mmol, 3 eq) in DMF (2 mL) were added HATU (541.73 mg, 1.42 mmol, 4 eq) and DIEA (184.14 mg, 1.42 mmol, 248.16 μL, 4 eq). After 0.5 h at 25° C. 2-(4-piperidylamino)-3H-quinazolin-4-one (100 mg, 356.19 μmol, 1 eq, HCl) was added. The mixture was stirred at 25° C. for 12 h. The reaction mixture was poured into water (10 mL). The mixture was extracted with EA (15 mL×3). The organic layers were combined, washed with brine (20 mL), dried over anhydrous Na₂SO₄, concentrated in vacuum. The product was purified by reverse phase prep-HPLC. Compound 11-1 (19.0 mg, 43.31 μmol, 12.16% yield, 95.4% purity) was obtained as a white solid. 1H NMR (400 MHZ, DMSO-d₆) δ 7.91; (br d, J=7.8 Hz, 1H), 7.64; (br s, 1H), 7.32; (br d, J=6.4 Hz, 1H), 7.21; (br s, 1H), 7.15-7.04; (m, 2H), 6.89; (br d, J=8.4 Hz, 2H), 4.23; (br s, 1H), 3.95; (br s, 1H), 3.73; (s, 3H), 3.03-2.81; (m, 2H), 2.05-1.59; (m, 2H), 1.38-0.99; (m, 6H). LCMS-ESI (m/z) calculated for C₂₄H₂₆N₄O₃: 418.20; found 419.2 [M+H]⁺, t_{R}=1.326 min (Method 2).

### Example 10

### MRGPRD Agonist Activity

Chinese Hamster Ovary (CHO) cells stably transfected to express human MRGPR D were maintained in an incubator at 37° C. with 5% CO₂ and grown in Ham's F-12 Nutrient Mix with 10% fetal bovine serum (FBS), 300 μg/mL Hygromycin B, 800 μg/mL Geneticin, and 1% each of Glutamax and penicillin/streptomycin. CHO cells stably transfected to express mouse MRGPR D were maintained in the same incubator and grown in Ham's F-12K (Kaighn's) media with 10% FBS, 500 μg/mL Geneticin, and 1% penicillin/streptomycin.

Cells were plated in a 384-well assay plate at 20,000 cells per well in 12 μL of Opti-MEM and kept in an incubator overnight. On the day of the assay, compounds solubilized at 10 mM in DMSO were added as a 10-point curve (30 μM final top concentration with 1:2 serial dilutions) using a Tecan D300E digital dispenser. Final concentrations of DMSO were kept consistent across the plate. 2 μl of assay buffer (final concentrations of 5.7 mM Tris-HCl, 43 mM NaCl, 50 mM LiCl, pH=8) are added to each well for a final assay volume of 14 μL. For a positive control β-alanine was solubilized in assay buffer and 2 μL are added to control wells in place of assay buffer alone. Plates were incubated in the dark for 1 h at 37° C. and then for 1 hour at room temperature. IP-1 standards and HTRF detection reagents were added according to the IP-One—Gq Kit purchased from Cisbio (part number 62IPAPEJ) and incubated in the dark for 1 h at room temperature. The plate was read on a Molecular Devices SpectraMax iD5 plate reader. The HTRF ratio was calculated from the raw data and graphed using GraphPad Prism to calculate an EC50 value for each compound.

Activity data for selected MRGPR D agonists are displayed in Table 10. The activity ranges are denoted as follows: “+++++” denotes agonist activity <100 nM; “++++” denotes agonist activity between 100 and 500 nM; “+++” denotes activity between 500 and 1000 nM; “++” denotes activity between 1000 and 5000 nM; and “+” denotes activity >5000 nM.

TABLE 10

| | MRGPR D Agonist |
|---|---|
| Cpd | Activity |
| No | IC₅₀ (nM) |
| 1-1 | ++ |
| 1-2 | ++ |
| 1-3 | + |
| 1-4 | ++ |
| 1-5 | ++ |
| 1-6 | + |
| 1-7 | ++ |
| 1-8 | + |
| 1-9 | + |
| 1-10 | +++ |
| 1-11 | + |
| 2-1 | + |
| 2-2 | + |
| 2-3 | + |
| 3-1 | + |
| 4-1 | ++ |
| 4-2 | + |
| 4-3 | ++ |
| 4-4 | ++ |
| 4-5 | + |
| 5-1 | + |
| 6-1 | ++ |
| 7-1 | ++ |
| 8-1 | ++ |
| 8-2 | ++ |
| 8-3 | + |
| 8-4 | + |
| 8-5 | ++ |
| 8-6 | ++++ |
| 8-7 | ++++ |
| 9-1 | +++ |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the structure of Formula (IIa): 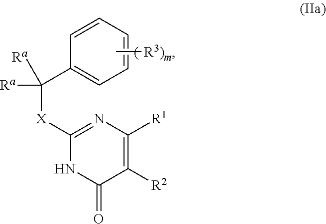
or a pharmaceutically acceptable isomer, racemate, hydrate, solvate, isotope, or salt thereof, wherein:
(A) X is NH;
R¹ is methyl;
R² is n-butyl;
R³ is CN, alkyl, alkoxy, or aminyl; and
either (1) each Rª is independently H or alkyl and m is 1, or (2) at least one Ra is alkyl and the other Ra is H or alkyl and m is 0 or 1; or
(B) X is O;
R¹ is alkyl and R² is alkyl, or R¹ and R² join together to form 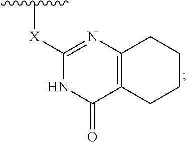
R³ is CN, alkyl, alkoxy, or aminyl;
each R^{a} is independently H or alkyl; and
m is 0 or 1.

2. The compound of claim 1, wherein X is O and R¹ and R² are both alkyl.

3-4. (canceled)

5. The compound of claim 1, wherein X is O and R¹ and R² join together to form

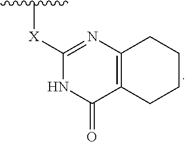

6. The compound of claim 1, wherein X is NH.

7-17. (canceled)

18. A compound having the structure of Formula (IIIa): 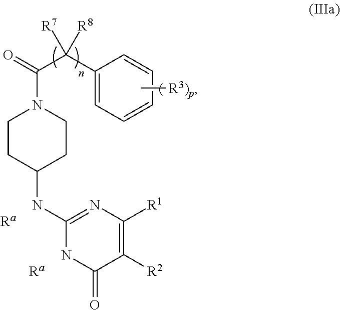
or a pharmaceutically acceptable isomer, racemate, hydrate, solvate, isotope, or salt thereof, wherein:
(A) R¹ is H and R² is H, halo, or alkyl; or R¹ and R² join together to form 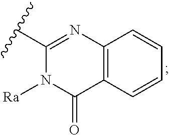
R³ is CN, alkyl, alkoxy, or aminyl;
each R⁷ and R⁸ are independently H or alkyl, or R⁷ and R⁸ when attached to the same carbon join together to form a cycloalkyl ring;
n is 0, 1, or 2; and
p is 0 or 1; or
(B) R¹ and R² join together to form 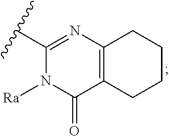
R³ is CN, alkyl, or alkoxy;
each R⁷ and R⁸ are independently H or alkyl;
n is 0, 1, or 2; and
p is 0 or 1.

19. The compound of claim 18, wherein both R¹ and R² are H.

20. The compound of claim 18, wherein R¹ is H and R² is halo.

21-22. (canceled)

23. The compound of claim 18, wherein R¹ is H and R² is alkyl.

24-25. (canceled)

26. The compound of claim 18, wherein R¹ and R² join together to form

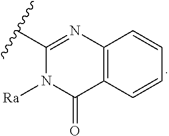

27. The compound of claim 18, wherein R¹ and R² join together to form

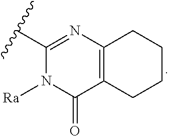

28-56. (canceled)

57. A compound having the structure of Formula (IVa): 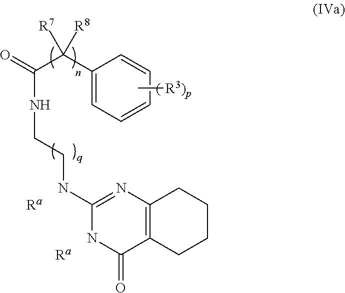
or a pharmaceutically acceptable isomer, racemate, hydrate, solvate, isotope, or salt thereof, wherein:
R³ is CN, alkyl, alkoxy, or aminyl;
each R⁷ is independently H or alkyl, each R⁸ is independently H or alkyl, or R⁷ and R⁸ are attached to the same carbon and join together to form a cycloalkyl ring;
each R^{a} is independently H or alkyl;
n is 1-2;
p is 0 or 1; and
q is 1-2.

58. The compound of claim 57, wherein R³ is CN.

59. The compound of claim 57, wherein R³ is alkyl.

60-61. (canceled)

62. The compound of claim 57, wherein R³ is alkoxy.

63-64. (canceled)

65. The compound of claim 57, wherein R³ is aminyl.

66-88. (canceled)

89. A compound having one of the following structures, or a pharmaceutically acceptable isomer, racemate, hydrate, solvate, isotope, or salt thereof:

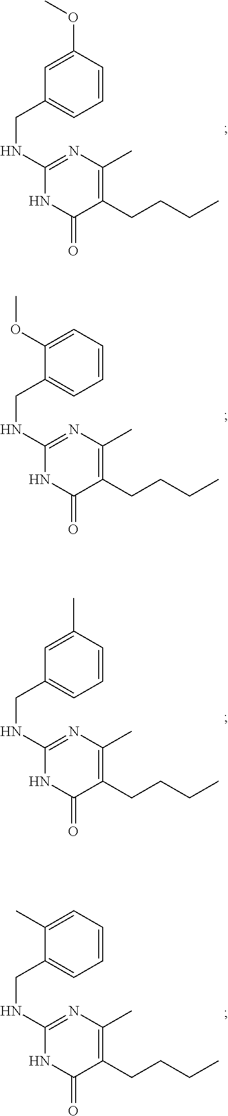 [SMILES: CCCCc1c(C)nc(NCc2cccc(OC)c2)[nH]c1=O ; CCCCc1c(C)nc(NCc2ccccc2OC)[nH]c1=O ; CCCCc1c(C)nc(NCc2cccc(C)c2)[nH]c1=O ; CCCCc1c(C)nc(NCc2ccccc2C)[nH]c1=O]

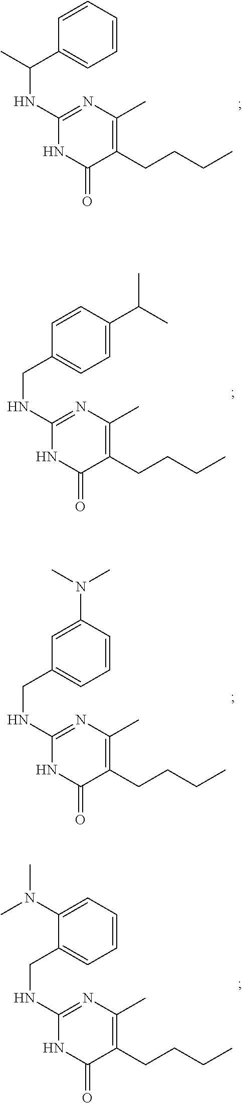

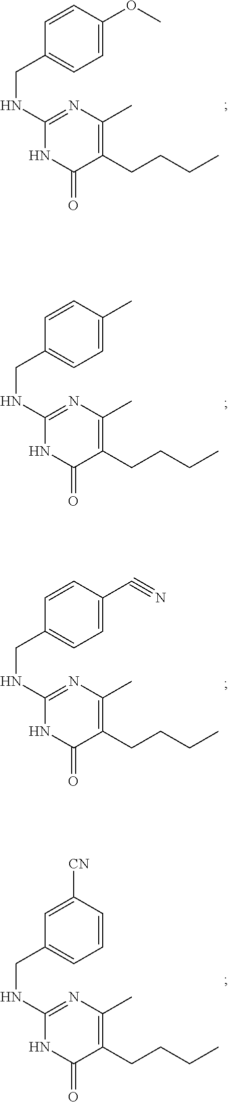 [SMILES: CCCCc1c(C)nc(NCc2ccc(OC)cc2)[nH]c1=O ; CCCCc1c(C)nc(NCc2ccc(C)cc2)[nH]c1=O ; CCCCc1c(C)nc(NCc2ccc(C#N)cc2)[nH]c1=O ; CCCCc1c(C)nc(NCc2cccc(C#N)c2)[nH]c1=O]

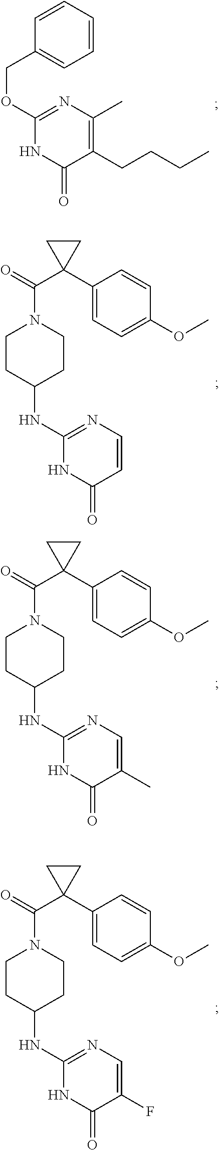

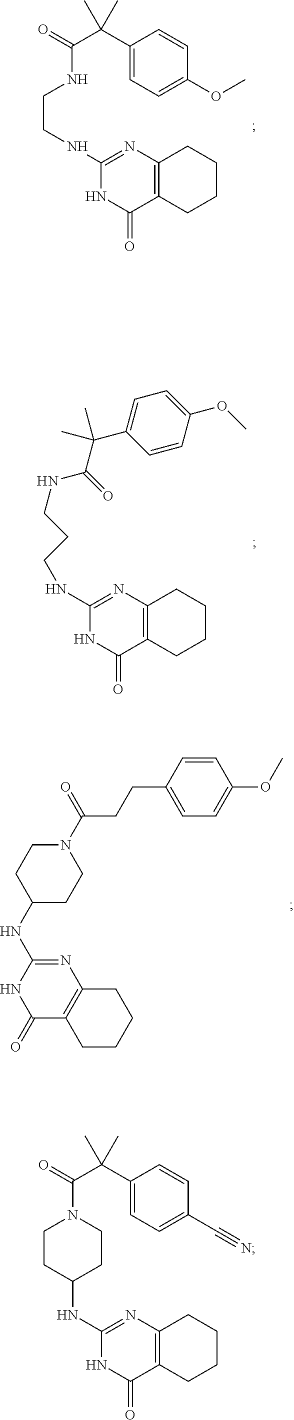 [SMILES: COc1ccc(C(C)(C)C(=O)NCCNc2nc3c(c(=O)[nH]2)CCCC3)cc1 ; COc1ccc(C(C)(C)C(=O)NCCCNc2nc3c(c(=O)[nH]2)CCCC3)cc1 ; COc1ccc(CCC(=O)N2CCC(Nc3nc4c(c(=O)[nH]3)CCCC4)CC2)cc1 ; CC(C)(C(=O)N1CCC(Nc2nc3c(c(=O)[nH]2)CCCC3)CC1)c1ccc(C#N)cc1]

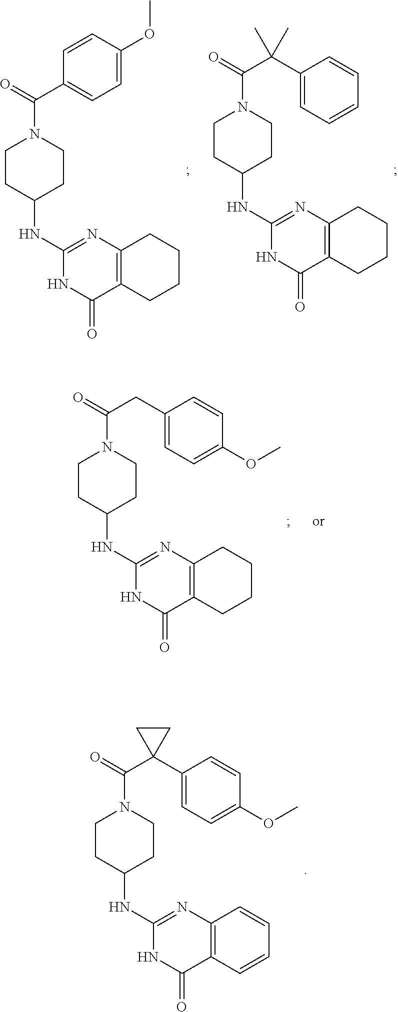 [SMILES: COc1ccc(C(=O)N2CCC(Nc3nc4c(c(=O)[nH]3)CCCC4)CC2)cc1 ; CC(C)(C(=O)N1CCC(Nc2nc3c(c(=O)[nH]2)CCCC3)CC1)c1ccccc1 ; COc1ccc(CC(=O)N2CCC(Nc3nc4c(c(=O)[nH]3)CCCC4)CC2)cc1 ; COc1ccc(C2(C(=O)N3CCC(Nc4nc5ccccc5c(=O)[nH]4)CC3)CC2)cc1]

90. A pharmaceutical composition comprising the compound of claim 1, or a pharmaceutically acceptable isomer, racemate, hydrate, solvate, isotope, or salt thereof and at least one pharmaceutically acceptable excipient.

91. A method of modulating a Mas-Related G-Protein Receptor (MRGPR) D by contacting MRGPRD with an effective amount of the compound of claim 1.

92. A method of treating a MRGPRD dependent condition by administering to a subject in need thereof an effective amount of the pharmaceutical composition of claim 90.

93. The method of claim 92, wherein the MRGPRD dependent condition is a pain associated condition, an itch associated condition, an ocular associated condition, a cardiovascular and renal disease associate condition, an inflammatory or autoimmune disorder, a cognitive impairment associated condition, a cancer related condition, or a non-cancerous hyperproliferative disorder.

94-95. (canceled)

96. The method of claim 93, wherein the itch associated condition is urticaria, pruritus, atopic dermatitis, dry skin, psoriasis, contact dermatitis, or eczema.

97-101. (canceled)

102. The method of claim 93, wherein the cancer related condition is lung cancer, pancreatic cancer, or skin cancer.

103-105. (canceled)

106. The method of claim 102, wherein the skin cancer related condition is melanoma.

107. The method of claim 93, wherein the non-cancerous hyperproliferative disorder is psoriasis, restenosis, or benign prostatic hypertrophy.
